(19) United States
(12) Patent Application Publication
(10) Pub. No.: US 2005/0191651 A1
(43) Pub. Date: Sep. 1, 2005

(54) Temperature-jump enhanced electrochemical detection of nucleic acid hybridization

(75) Inventors: Stefan Franzen, Apex, NC (US); Daniel Feldheim, Cary, NC (US); Scott H. Brewer, Danville, VA (US); Lisa B. Lowe, Clayton, NC (US)

Correspondence Address: JENKINS, WILSON & TAYLOR, P. A., 3100 TOWER BLVD, SUITE 1400, DURHAM, NC, 27707, US

(73) Assignee: North Carolina State University

(21) Appl. No.: 10/978,756

(22) Filed: Nov. 1, 2004

Related U.S. Application Data
(60) Provisional application No. 60/515,920, filed on Oct. 30, 2003.

(51) Int. Cl. C12Q 1/68

(52) U.S. Cl. 435/6

(57) ABSTRACT

A nucleic acid hybridization detection assay is carried out at a solid electrode. A solid electrode, such as an indium tin oxide electrode, is modified by single-stranded capture oligonucleotides that are immobilized to the surface of the electrode. Using sandwich assay methodology, complementary target nucleic acid sequences hybridize to the capture oligonucleotides, which are in turn hybridized to a detection probe comprising a nanoparticle. When the assay is carried out in the presence of a redox mediator in solution, the nanoparticle catalyzes the transfer of electrons to the electrode, thus generating a detectable electrical current.

### CROSS-REFERENCE TO RELATED APPLICATIONS

This application claims the benefit of and priority to U.S. Provisional Patent Application Ser. No. 60/515,920, filed Oct. 30, 2003, the disclosure of which is incorporated herein by reference in its entirety.

### TECHNICAL FIELD

The presently described methods relate to electrochemical detection of biological molecules such as nucleic acids. In particular, the methods described herein relate to the thermal enhancement of electrochemical detection of nucleic acid hybridization.

### BACKGROUND

The detection of specific nucleic acid sequences in biological samples provides a basis for myriad practical and research techniques, including gene identification, mutation detection, gene expression profiling, and DNA sequencing. Diagnostic and forensic applications are but two areas in which nucleic acid detection techniques find widespread use.

Particular nucleic acid sequences are usually detected by one or more nucleic acid hybridization assays, in which the presence of a target sequence in a biological sample is determined by hybridizing a probe sequence designed to specifically bind the target with heterogeneous nucleic acids in the sample. The presence of the target is usually indicated by the detection of a chemical, enzymatic, magnetic or spectroscopic label that is directly or indirectly attached to either the probe or the target sequence. Such hybridization assays are increasingly being combined with parallel, high-throughput microarray technology, in which thousands of hybridization assays are carried out simultaneously on a solid substrate (e.g., a “chip”). Microarray technologies are highly amenable to automation and facilitate the screening of, for example, one biological sample against a large number of probes in a brief time period.

A broad spectrum of labeling and detection methodologies are currently used in conjunction with nucleic acid hybridization and microarray techniques. When labeled probes are used, for example, the presence of a target sequence in a biological sample is usually determined by separating hybridized and non-hybridized probe, and then directly or indirectly measuring the amount of labeled probe that is hybridized to the target. Suitable labels can provide signals detectable by luminescence, radioactivity, colorimetry, x-ray diffraction or absorption, magnetism or enzymatic activity, and can include, for example, fluorophores, chromophores, radioactive isotopes, light-scattering particles, magnetic particles, enzymes, and ligands having specific binding partners. The specific labeling method chosen depends on a multitude of factors, such as ease of attachment of the label, its sensitivity and stability over time, speed and ease of detection and quantification, and cost and safety factors.

Despite the abundance of labeling techniques, the utility, versatility and diagnostic value of any particular system for detecting nucleic acid sequences of interest can be limited. For example, fluorescent labeling and detection methodologies are generally not sufficiently sensitive to single-base mismatches in surface-bound hybridization duplexes. Additionally, fluorescence-based techniques require extensive sample preparation, as well as the use of unwieldy apparatus such as confocal microscopes. Moreover, many commonly used labeling and detection techniques have undesirably high limits of detection, thus necessitating the use of costly and time-consuming nucleic acid amplification techniques. Sensitive methods that are able to differentially detect very low concentrations of target nucleic acids thus remain in demand.

### SUMMARY

Described herein are sensitive, electrochemical methods for detecting nucleic acid sequences and nucleic acid hybridization events.

In some embodiments provided are methods of detecting a target nucleic acid sequence, comprising: providing a hybridization complex comprising (a) a capture probe that is attached to an electrode and (b) a target nucleic acid sequence that is hybridized to the capture probe, wherein the target nucleic acid sequence additionally comprises at least one nanoparticle attached to the target nucleic acid sequence; exposing the electrode to light while the electrode is in contact with a redox solution, wherein the redox solution comprises a redox mediator and an electrolyte, and wherein the light has a wavelength absorbed by the nanoparticle; and detecting an electrical signal in the electrode, whereby detection of an increased electrical signal relative to a signal that would be detected in the absence of said complex indicates the presence or amount of target nucleic acid sequence hybridized to the electrode. In some embodiments a detection probe is not employed.

In some embodiments, a target nucleic acid sequence hybridizes a capture oligonucleotide probe that is attached to the surface of an electrode. The target sequence is then hybridized with a detection probe comprising a nanoparticle, thus forming a capture probe-target sequence-detection probe hybridization complex, or “sandwich”. In the presence of a redox solution comprising a redox mediator, the hybridization complex is exposed to light (e.g., as generated by a laser) at a wavelength that is absorbed by the nanoparticle and causes the nanoparticle to generate heat. In some embodiments, the light wavelength matches the surface plasmon resonance of the nanoparticle. The light excitation of the nanoparticle elicits a temperature jump in the environment surrounding the nanoparticle, while the redox mediator facilitates electron transfer to the surface of the electrode. The electron transfer generates a detectable electrical current in the electrode, the sensitivity of which detection is significantly enhanced by the light-induced temperature jump. The detected electrical current provides a measure of nucleic acid hybridization at the surface of the electrode, which can be correlated with the concentration of target nucleic acid present in the sample.

In some embodiments, target sequences and capture probes comprise single-stranded nucleic acid regions, while detection probes comprise a nanoparticle and an oligonucleotide. In some embodiments, the detection probe comprises a nanoparticle and one partner of a ligand-binding pair (e.g., streptavidin), while the target nucleic acid comprises the other partner of the ligand-binding pair of the detection probe (e.g., biotin). In some embodiments, the target sequence is labeled with biotin during an amplification reaction in which RNA is used as a template and nucleotides labeled with biotin are incorporated into a complementary cDNA strand using reverse transcriptase.

In some embodiments, the electrode and nanoparticles used in the described methods comprise different materials that are each selected from the group consisting of metals and metal oxides. In some embodiments, the electrode comprises indium tin oxide, and the nanoparticle comprises gold (Au).

In some embodiments, the electrical current in the electrode is detected using voltammetry. In another embodiment, the electrical current in the electrode is detected using chronoamperometry.

Thus, the presently disclosed subject matter provides a method of detecting nucleic acid hybridization. Accordingly, the detection of nucleic acid hybridization is achieved in whole or in part by the methods described in more detail below.

### BRIEF DESCRIPTION OF THE DRAWINGS

FIGS. 1A and 1B are schematic diagrams illustrating an electrochemical detection strategy. FIG. 1A is an illustration of an indium tin oxide electrode used in the present methods, to which a plurality of capture probes has been attached. An indium tin oxide electrode is shown for the purposes of illustration only, and not for the purposes of limitation. A gold nanoparticle is shown for the purposes of illustration only, and not for the purposes of limitation. FIG. 1B illustrates an embodiment of the present methods in which a target cDNA has hybridized to capture probes attached to the electrode surface shown in FIG. 1A. A detection probe comprising a gold nanoparticle and an oligonucleotide has hybridized to the target cDNA; accordingly, a hybridization complex comprising a capture probe, target sequence and detection probe is illustrated. The surface of the electrode, with the hybridization complex attached, is in the presence of an electrolyte solution comprising a redox molecule. Irradiation with light causes the gold nanoparticles to heat, which increases the temperature of the solution surrounding the nanoparticles. The increase in temperature drives the redox reaction, which generates an electron.

FIG. 2 is an absorbance spectrum of a gold nanoparticle as a function of increasing light wavelength. When the gold nanoparticle is irradiated at 532 nm (near the surface plasmon resonance of gold), a jump in absorbance is observed.

FIG. 3 is a schematic diagram of illustrating a method by which nucleic acid molecules can be attached to a nanoparticle.

FIG. 4 illustrates the formation of an amide bond by the activation of the carboxylic acid on a monolayer of 12-phosphonododecanoic acid on ITO by EDC with 5′ modified C₃NH₂ ssDNA. ITO is shown for the purposes of illustration only, and not for the purposes of limitation.

FIG. 5 is an x-ray photoelectron spectra (XPS) of In 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

FIG. 6 is an XPS spectra of Sn 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

FIG. 7 is an XPS N 1s spectra of ITO modified with a monolayer of 12-phosphonododecanoic acid (long dash) and ITO modified with ssDNA coupled through a monolayer of 12-phosphonododecanoic acid (short dash) fitted to a Gaussian line shape (solid).

FIG. 8 is an XPS Au 4f_{7/2,5/2} spectra of ITO modified with ssDNA coupled through a monolayer of 12-phosphonododecanoic acid (dotted line) exposed to the complementary (short dash) or non-complementary (long dash) ssDNA labeled with a 10 nm gold nanoparticle (1 nM) fitted to two Gaussian line shapes (solid).

FIG. 9 is a grazing angle reflectance FTIR spectra of ITO modified with a monolayer of 12-phosphonododecanoic acid (solid) coupled to ssDNA (dashed) recorded at an incident angle of 80 degrees with p-polarized radiation.

FIG. 10 is a graphical illustration of anodic current vs. time for ITO electrodes in (A) 0.1 M phosphate buffer (pH 7.3), (B) 0.1 M phosphate buffer/0.05 M EDTA, and (C) 0.1 M phosphate buffer following adsorption of 10 nm diameter gold particles to the electrode via aminosilane linkers. The current trace shown in (D) illustrates the conditions of the electrode from (C), when 0.05 M EDTA added to solution. The arrow indicates the start of an approximately 15 second laser irradiation cycle with 532 nm light (0.64 W/cm²). The potential was held at 0.3 V vs. Ag₍ₛ₎/AgCl.

FIG. 11 shows a cyclic voltammogram (left) and a graph of photocurrent vs. applied potential (right) for EDTA on gold-nanoparticle-coated ITO electrodes.

FIG. 12 is a graphical illustration of open circuit voltage vs. time for ITO electrodes in contact with 100 mM ferrocene and 0.1 mM ferrocinium in acetonitrile/0.1 M NaClO₄. The electrode in the top curve contained 1.5×10¹⁰ gold nanoparticles cm⁻². The bottom curve was ssDNA-coated ITO. Downward and upward arrows indicate light on and off, respectively. In FIG. 12, the curves are offset for clarity.

FIG. 13 illustrates the laser-induced temperature jump effect as manifested on a gold-nanoparticle-coated ITO electrode. FIG. 13 is a graph showing an increase in electrode temperature as a function of time, when the electrode is an ITO electrode coated with gold nanoparticles attached to the electrode surface with oligonucleotides, and when the nanoparticles are irradiated with a YAG laser at 532 nm. An indium tin oxide electrode is employed for the purposes of illustration only, and not for the purposes of limitation. A gold nanoparticle is employed for the purposes of illustration only, and not for the purposes of limitation.

FIG. 14 shows a series of infrared thermograms (8 μm-12 μm) of gold nanoparticle-coated glass slides under irradiation with 532 nm light (16 W/cm²). Particle densities were 1×10¹⁰ cm⁻², 2×10¹⁰ cm⁻², and 3.5×10¹⁰ cm⁻² for A, B, and C, with recorded temperatures of 30.5° C., 35.3° C., and 42.9° C., respectively. Light-off temperature was 24.6° C. (ΔT for bare glass was <2° C.).

FIG. 15 is an illustration of anodic current vs. time for an ITO electrode in 0.1 M phosphate buffer/0.05 M EDTA following adsorption of 10 nm diameter gold particles to the electrode via DNA hybridization. The potential was held at 0.5 V vs. Ag/AgCl. The bottom current trace represents ssDNA/gold nanoparticle conjugates hybridized from a 100 fM solution. The top trace represents ssDNA probe strands on ITO. The Arrow indicates light on. The current signal in the bottom trace is ˜2×background current of top trace.

FIG. 16 is an illustration of the limits of detection of methods of the presently disclosed subject matter. The striped points indicate background current, while solid points represent the detected current as a function of concentration in (pM) of ss-DNA-conjugated gold nanoparticles. The present methods can are able to detect (distinguish over background) hybridization of nucleic acids at electrode surfaces in concentrations as low as 0.1 pM.

FIG. 17 is a graph comparing the cyclic voltammogram trace of gold nanoparticles hybridized onto ITO electrodes when the electrode solution comprises an electrolyte solution without EDTA (KP, upper trace/small current peak observed) and with EDTA (KP/EDTA, lower trace/large current peak observed).

FIGS. 18A and 18B, taken together, provide a graphical comparison of a known method of incorporating a fluorescent label into a target nucleic acid (FIG. 18A) and a presently described method of incorporating one partner of ligand binding pair (e.g., biotin) into a target nucleic acid, which ligand binding pair partner can then be used to bind a nanoparticle to which is attached the other member of the ligand binding pair (e.g., streptavidin) (FIG. 18B).

FIG. 19 is a plot of a thermographic excitation profile for 12- to 15-nm gold nanoparticles. A gold nanoparticle is shown for the purposes of illustration only, and not for the purposes of limitation. More particularly, FIG. 19 illustrates that the heat released from a gold nanoparticle can be directly related to the absorbance spectrum of the gold nanoparticle. The Figure also demonstrates that exciting a gold nanoparticle near the absorption maximum can yield the highest temperature change. The solid line represents the UV-VIS spectrum for 12- to 15-nm gold nanoparticles and the solid triangles (▴) represent the thermographic excitation profile for 12- to 15-nm gold nanoparticles.

FIG. 20 is a plot showing that in the thermographic detection of nucleic acids, the temperature increase (in ° C.) after 30 seconds of illumination can be a linear function of the nanoparticle density, i.e., the amount of nanoparticles per spot (in amole plotted in log scale), over many orders of magnitude. For example, the linear fit (R²) of the data provided in FIG. 20 is 99.8%.

FIG. 21 is a plot showing an influence of laser power on the kinetics of the thermographic detection of nucleic acids. More particularly, FIG. 21 shows how quickly (time in seconds) the temperature rises (in ° C.) for a number of laser powers. The conditions under which the data presented in FIG. 21 were obtained are as follows: 10 nm citrate-coated gold nanoparticles; 9.5 fmole nanoparticles per spot; spot size=2 mm in diameter; beam diameter=2 mm. The laser was a Coherent Verdi V-10 CW laser operating at 532 nm (Coherent, Inc., Santa Clara, Calif., United States of America). Legend: (♦)=1.0 W laser power; (▪)=0.75 W laser power; ( [symbol] ) 0.50 W laser power; and ( [symbol] ) 0.25 W laser power. FIG. 22 is a plot illustrating the reversibility of the thermographic effect in the thermographic detection of nucleic acids. The conditions under which the data presented in FIG. 22 were obtained are as follows: 10 nm citrate-coated gold nanoparticles; 0.5 fmole nanoparticles per spot; spot size=2 mm in diameter; beam diameter=2 mm; the laser was a Coherent Verdi V-10 CW laser operating at 532 nm with a laser power of 0.75 W.

FIG. 23 is a digital image showing that arrays can be read with thermography. More particularly, FIG. 23 illustrates the bloc reading of a 3×3 array. The conditions under which the data presented in FIG. 23 were obtained are as follows: 10 nm citrate-coated gold nanoparticles; 0.475 fmole nanoparticles per spot; spot size=2 mm in diameter; spot-to-spot spacing=2 mm; the laser was a Coherent Verdi V-1 0 CW laser operating at 532 nm with a laser power of 3 W with a beam diameter of 1 cm.

### DETAILED DESCRIPTION

The present disclosed subject matter will now be described more fully hereinafter with reference to the accompanying Examples and Figures, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Unless otherwise defined, all technical and scientific terms used herein have the same meaning as commonly understood by one of ordinary skill in the art to which the presently disclosed subject matter belongs. All publications, patent applications, patents, and other references mentioned herein are incorporated by reference in their entirety. Throughout the specification and claims, a given chemical formula or name shall encompass all optical and stereoisomers as well as racemic mixtures where such isomers and mixtures exist.

I. General Considerations

Surface plasmon resonance (SPR) is a quantum optical-electrical phenomenon arising from the interaction of light with a metal surface. Under certain conditions, the energy carried by photons of light is transferred to packets of electrons, called plasmons, on a metal's surface. Energy transfer occurs only at a specific resonance wavelength of light, specifically, at the wavelength where the quantum energy carried by the photons exactly equals the quantum energy level of the plasmons.

Resonance wavelength can be determined very precisely by measuring the light reflected by a metal surface. At wavelengths longer than the plasmon wavelength (or frequencies lower than the plasmon frequency), the metal acts as a mirror, reflecting virtually all the incident light. At the wavelength that fulfills the resonance conditions, the incident light is almost completely absorbed. The wavelength at which maximum light absorption occurs is the resonance wavelength.

The coupling of light into a metal surface results in the creation of a plasmon, a group of excited electrons which behave like a single electrical entity. The plasmon, in turn, generates an electrical field which extends about 100 nanometers above the metal surface. One characteristic of SPR that makes it a useful analytical tool is that any change in the environment within the range of the plasmon field causes a change in the wavelength of light that resonates with the plasmon. Stated otherwise, an environmental alteration results in a shift in the wavelength of light that is absorbed rather than reflected. The magnitude of the shift is generally quantitatively related to the magnitude of the alteration.

Nanometer-sized metal particles such as gold and silver have intense plasmon resonances in the visible region of the electromagnetic spectrum. Excitation of gold nanoparticle plasmons causes rapid local temperature changes, which have been used previously to induce polymer gel swelling transitions (see, e.g., C. D. Jones and L. A. Lyon, J. Am. Chem. Soc. (2003) 125, 460, and S. R. Sershen et al., J. Biomed. Maer. Res. (2000) 51, 293), and to study the dynamics of melt transitions in nanometer-sized metals (see, e.g., S. Link et al., J. Phys. Chem. B (2000) 104, 6152).

Temperature jumps at an electrode-solution interface alter the open-circuit potential of the electrode vs. a reference electrode via primarily four mechanisms: (i) a junction potential induced between the hot electrode and the cold contact; (ii) a junction potential induced between the hot electrical double layer and cold bulk solution (the Soret effect); (iii) a change in the electrical potential (ΔV_{dl}) of the electrode relative to the outer Helmholtz plane due to perturbed equilibria involving ions and solvent dipoles; and (iv) a redox potential change (ΔV_{redox}) due to the presence of electron donors and acceptors in solution. The dominant effects are the latter two. Laser-induced temperature jumps involving gold nanoparticle plasmon excitations are manifest as open-circuit photovoltage changes and photoelectrochemical currents.

II. Thermally-Enhanced Electrochemical Detection of Nucleic Acid Hybridization

A. General Overview of Methods

The methods described herein are useful for detecting nucleic acid hybridization events. More particularly, the present methods are useful for detecting specific target nucleic acid sequences in a heterogeneous sample.

In some embodiments provided are methods of detecting a target nucleic acid sequence, comprising: providing a hybridization complex comprising (a) a capture probe that is attached to an electrode and (b) a target nucleic acid sequence that is hybridized to the capture probe, wherein the target nucleic acid sequence additionally comprises at least one nanoparticle attached to the target nucleic acid sequence; exposing the electrode to light while the electrode is in contact with a redox solution, wherein the redox solution comprises a redox mediator and an electrolyte, and wherein the light has a wavelength absorbed by the nanoparticle; and detecting an electrical signal in the electrode, whereby detection of an increased electrical signal relative to a signal that would be detected in the absence of said complex indicates the presence or amount of target nucleic acid sequence hybridized to the electrode. In some embodiments a detection probe is not employed.

In some embodiments, a nucleic acid hybridization detection assay is carried out at a solid electrode surface. A solid electrode, such as an indium tin oxide electrode, is modified by single-stranded capture oligonucleotide probes that are immobilized to the surface of the electrode. Using sandwich assay methodology, the capture probes hybridize with complementary target nucleic acid sequences, which are in turn hybridized by a detection probe comprising a nanoparticle-oligonucleotide conjugate. Thus, the target sequence forms part of a hybridization complex comprising a capture probe, a target sequence, and a detection probe.

As used herein, the terms “complex”, “duplex,” and “hybridization complex” are used interchangeably, and mean a structure formed of at least two different members. Hybridization complexes can comprise two or more DNA sequences, RNA sequences or combinations thereof. Complexes, in general, form via hybridization of complementary strands of nucleic acids (e.g., by Watson Crick or Hoogsteen base-pairing), e.g., DNA or RNA. A member of a hybridization complex can itself comprise one, two or more members. Thus a complex can comprise a structure comprising two members, one or both of which can itself be a complex. For example, one member of a complex can comprise a single stranded nucleic acid sequence (immobilized or in solution) and the second member of the complex can comprise a nucleic acid double stranded complex (immobilized or in solution), effectively making the complex a triplex structure.

The term “target sequence,” as used herein, means a nucleic acid sequence on a single strand of nucleic acid. A target sequence can accordingly be a single-stranded segment of a target nucleic acid. If the target nucleic acid is single-stranded, the target sequence can be identical to the target nucleic acid, or can comprise a portion or sub-sequence of the target nucleic acid. If the target nucleic acid is double-stranded DNA, the target sequence can be identical to or comprise a sub-sequence of the coding strand, or can be identical to or comprise a sub-sequence of the anti-parallel, complementary, non-coding strand. As described in further detail below, target sequences can optionally comprises additional moieties such as labels or tags, which facilitate binding to a detection probe comprising a nanoparticle.

A “capture probe,” as used herein, is an oligonucleotide that will hybridize to a target nucleic acid sequence, and which is used to probe for the presence of the target sequence. The capture probe enables the attachment of a target nucleic acid to the solid electrode, for the purposes of detection. A “detection probe,” as used herein, comprises a nanoparticle, and typically comprises a nanoparticle-oligonucleotide conjugate. Thus, each probe typically comprises an oligonucleotide sequence attached to either a particle or a solid surface. In general, the capture probe is bound to an electrode surface, while the detection probe comprises an oligonucleotide attached to a nanoparticle.

Nanoparticles and electrodes of the presently disclosed subject matter can be fabricated from a broad range of materials, with one limitation being that the nanoparticle material and the electrode material are not identical. Moreover, the nanoparticle comprises a material that absorbs light at one or more particular frequencies (i.e., exhibits surface plasmon resonance or interband transition), while the electrode generally comprises a conductive material. Accordingly, nanoparticles and electrodes, as used in the present methods, typically comprise metal or metal oxide materials.

In some embodiments, the capture oligonucleotide probe hybridizes a first domain of the target sequence, while the oligonucleotide component of the detection probe hybridizes a second domain of the target sequence to form a hybridization complex. In other embodiments, the detection probe can bind to the same domain as the capture probe, forming a triplex.

Detection of the hybridization complex is facilitated by contacting the electrode surface with an electrolyte solution comprising a redox mediator, referred to herein as a “redox solution”. Contact with the redox solution can occur either concurrently with or subsequent to the formation of the sandwich hybridization complex. The nanoparticle component of the detection probe catalyzes electron transfer to the electrode surface, thus creating a detectable electrical current (e.g., a photocurrent) in the electrode.

In some embodiments, the wavelength of light used to photoexcite the nanoparticle matches the surface plasmon resonance of the nanoparticle, thus generating heat and eliciting a temperature jump in the environment immediately surrounding the nanoparticle and the hybridization complex. In another embodiment, the wavelength of light does not match the surface plasmon resonance of the nanoparticle, but nonetheless is absorbed by the nanoparticle (e.g., due to interband transition of a metal nanoparticle), thus generating heat.

Comparing the difference between electrical current generated in the electrode by the photoinduced electron transfer and the electrical current generated by a bare reference electrode (i.e., an electrode unmodified by nanoparticles) provides a measure of nucleic acid hybridization at the electrode surface. Alternatively or in addition, comparing the difference in potential between the modified, irradiated electrode and the complex-free electrode provides a measure of nucleic acid hybridization at the electrode surface. This measure of hybridization can be correlated to the concentration of target nucleic acid in the sample. The limit of detection is enhanced by exciting the nanoparticle with light at a wavelength that is absorbed by the nanoparticle. The heat generated by the nanoparticle excitation enhances the electrochemical response. In accordance with experiments described herein, detection sensitivities on the order of about 100 fM or better of target ssDNA-modified nanoparticles at the electrode surface have been observed.

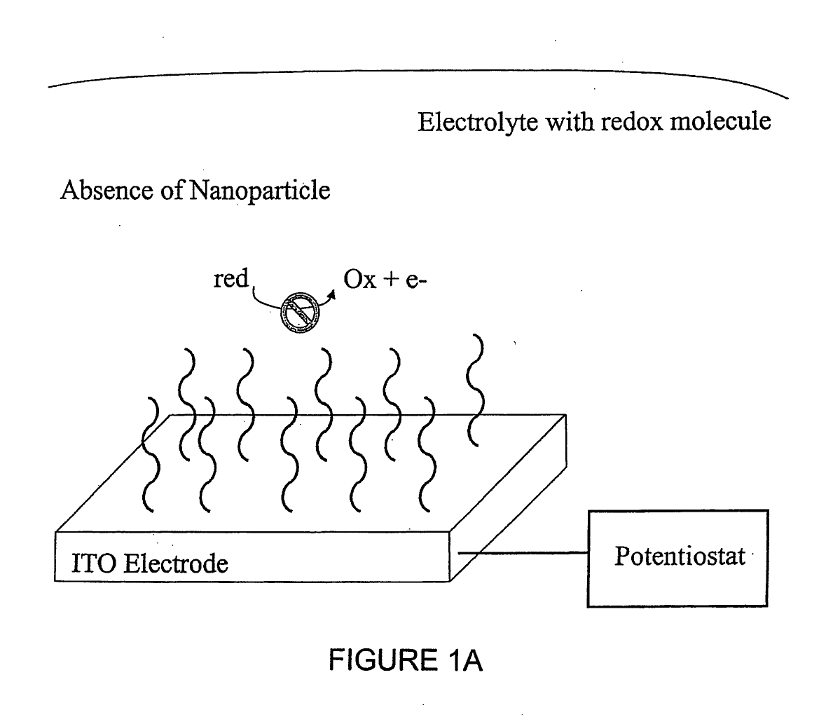

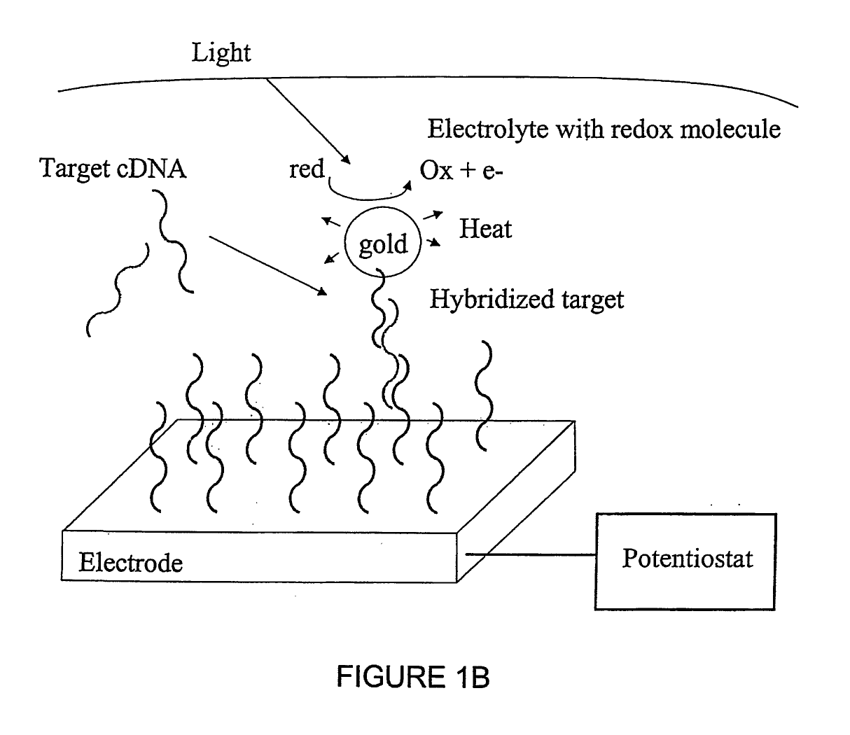

FIGS. 1A and 1B, taken together, provide a graphical illustration of the thermally enhanced, light-induced electrochemical interactions underlying the detection methods described herein. FIG. 1A is an illustration of an indium tin oxide electrode used in the present methods, to which a plurality of capture probes have been attached. Neither target sequences nor detection probes comprising nanoparticles have been brought into contact with the electrode, although the electrode is shown as being in the presence of an electrolyte solution comprising a redox molecule. As illustrated in the Figure, a redox reaction releasing an electron is not catalyzed in this scenario, due to the absence of a nanoparticle to serve as a catalyst for electron transfer to the electrode surface and the absence of light induction.

FIG. 1B illustrates an embodiment of the present methods in which a target cDNA is hybridized to capture probes attached to the electrode surface. A detection probe comprising a gold nanoparticle and an oligonucleotide is hybridized to the target cDNA; accordingly, a hybridization complex comprising a capture probe, target sequence and detection probe is illustrated. The electrode surface, with the hybridization complex attached, is in the presence of an electrolyte solution comprising a redox molecule. Light induction causes the gold nanoparticles to heat, increasing the temperature of the solution surrounding the nanoparticles. The increase in temperature drives the redox reaction, which generates an electron. In some embodiments, the electric current generated by the electron transfer to the electrode is measured with reference to controlled potential, which is controlled by a potentiostat, as shown in the Figure.

In some embodiments the presently disclosed methods are free of (i.e., do not involve) the use of a target analyte attached to a conductive support and/or a nanoparticle comprising a photoelectrochemically active moiety such as a ruthenium complex (e.g., ruthenium tris-bipyridine and related adducts that have long-lived excited states). In some embodiments the presently disclosed methods are free of a nanoparticle comprising a photoelectrochemically active moiety such as a ruthenium complex (e.g., ruthenium tris-bipyridine and related adducts that have long-lived excited states). In some embodiments the presently disclosed methods are free of a photoelectrochemically active moiety that performs a “dye” or label function in the practice of these methods. In some embodiments the presently disclosed subject matter does not expose a photoelectrochemically active moiety to light, wherein the light has a wavelength absorbed by the nanoparticle but that the light does not generate a photoelectric current between a photoelectrochemically active moiety and a conductive support (e.g., the wavelength of the light that is used to target the nanoparticle is different that a wavelength that would excite a photochemically active moiety).

B. Nucleic Acid Sequences

The methods described herein are useful for the detection of target nucleic acid sequences and nucleic acid hybridization events. Probes useful in the detection of target sequences and nucleic acid hybridization events comprise nucleic acids in the form of oligonucleotides.

As used herein, the terms “nucleic acid,” “nucleic acid sequence,” “nucleic acid molecule,” and grammatical equivalents mean at least two nucleotides covalently linked together. Nucleic acids can be single-stranded or double-stranded, as specified, or contain portions of both double-stranded or single-stranded sequence. Nucleic acids can comprise any combination of deoxyribo- and ribonucleotides, and any combination of bases, including uracil, adenine, thymine, cytosine, guanine, inosine, xathanine hypoxathanine, isocytosine, isoguanine, etc. Deoxyribonucleic acids (DNA) can comprise genomic DNA, cDNA derived from ribonucleic acid, DNA from an organelle (e.g., mitochondrial DNA or chloroplast DNA), synthesized DNA (e.g., oligonucleotides), or combinations thereof. Ribonucleic acids (RNA) can comprise genomic RNA (e.g., viral genomic RNA), messenger RNA (mRNA), ribosomal RNA (rRNA), transfer RNA (tRNA), or combinations thereof.

A nucleic acid of the presently disclosed subject matter will generally contain phosphodiester bonds, although in some cases, as outlined below, nucleic acid analogs are included that can have alternate backbones, comprising, for example, phosphoramide, phosphorodithioate, methylphophoroamidite linkages, and peptide nucleic acid backbones and linkages. Other analog nucleic acids include those with positively-charged backbones, non-ionic backbones and nonribose backbones. Nucleic acids containing one or more carbocyclic sugars are also included within the definition of nucleic acids. Mixtures of naturally occurring nucleic acids and analogs can be used. Alternatively or in addition, mixtures or chimeras of different nucleic acid analogs, and mixtures of naturally occurring nucleic acids and analogs can be used.

Peptide nucleic acids (PNA) are specifically included in the definition of nucleic acids, as used herein. PNAs are DNA analogs wherein the backbone of the analog (for example, a sugar backbone in DNA) is a pseudopeptide. A PNA backbone can comprise, for example, a sequence of repeated N-(2-amino-ethyl)-glycine units. A peptide nucleic acid analog reacts as DNA would react in a given environment, and can bind complementary nucleic acid sequences and various proteins. Peptide nucleic acid analogs offer the potential advantage over unmodified DNA of the formation of stronger bonds, due to the neutrally charged peptide backbone of the analogs, and can impart a higher degree of specificity than is achievable by unmodified DNA. These backbones are substantially non-ionic under neutral conditions, in contrast to the highly charged phosphodiester backbone of naturally occurring nucleic acids.

Nucleic acids can also comprise “locked nucleic acids”, also known as LNAs (e.g., WO 98/39352).

When used as oligonucleotide probes, as defined herein, nucleic acids can be analytically pure, as determined by spectrophotometric measurements or by visual inspection following electrophoretic resolution. In some embodiments, nucleic acids that are to be amplified can be preferably analytically pure, although purity is not a requirement. In certain desirable embodiments, nucleic acid samples are free of contaminants such as polysaccharides, proteins and inhibitors of enzyme reactions. When an RNA sample is intended for use as probe or target sequence, it is preferably free of DNAase and RNAase. Contaminants and inhibitors can be removed or substantially reduced using resins for DNA extraction or by standard phenol extraction and ethanol precipitation, as is taught in the art.

1. Target Nucleic Acids and Sequences

A target sequence can be selected on the basis of the context in which the present methods are employed. Target sequences can vary widely. For example, desirable target sequences include, but are not limited, to characteristic or unique nucleic acid sequences found in various microbes or mutated DNA that can be used in the diagnosis of diseases, in environmental bioremediation, in the determination of genetic disorders, and in genetic epidemiology. Functional equivalents of known sequences can also be used as target sequences.

The target sequence can comprise a portion of a gene, a regulatory sequence, genomic DNA, cDNA, RNA including mRNA and rRNA, or others. The target sequence can be a target sequence from a biological sample, as discussed herein, or can be a secondary target such as a product of an amplification reaction. The target sequence can take many forms. For example, a target can be contained within a larger nucleic acid sequence, i.e., all or part of a gene or mRNA, a restriction fragment of a plasmid or genomic DNA, among others. Target nucleic acids can be excised from a larger nucleic acid sample using restriction endonucleases, which sever nucleic acid sequences at known points in a nucleic acid sequence. Excised nucleic acid sequences can be isolated and purified by employing standard techniques. Target sequences can also be prepared by reverse transcription processes. See, e.g., Sambrook et al., Molecular Cloning: A Laboratory Manual (Cold Spring Harbor, N.Y. (1992)).

A target sequence can comprise one or more different target domains. A target domain is a contiguous, partial sequence (i.e., a sub-sequence) of the entire target sequence, and can be any nucleotide length that is shorter than the entire target sequence. In some embodiments, a first target domain of a target sequence hybridizes a capture probe, while a second and different target domain hybridizes an oligonucleotide component of a detection probe. Target domains can be adjacent or separated, as indicated. For example, a first target domain can be directly adjacent (i.e., contiguous) to a second target domain, or the first and second target domains can be separated by an intervening target domain. Assuming a 5′ to 3′ orientation of a target sequence, a first target domain can be located either 5′ to a second target domain, or 3′ to a second domain.

If desired, a target sequence can further comprise an additional moiety such as one partner of a ligand-binding pair, in order to facilitate binding to a detection probe comprising the other partner of the ligand binding pair attached to a nanoparticle. For example, the target sequence can comprise a biotin label or tag, which will facilitate binding to a detection probe comprising a nanoparticle attached to streptavidin. The biotin tag can be incorporated into the target sequence using amplification methods that are analogous to known methods used to incorporate fluorescent labels into target molecules, as set forth in more detail below.

Nucleic acid sequences of any practical length can be used as a target sequence. Generally, a target sequence is between ten and 50 nucleotides in length, and thus target sequences of ten, 15, 20, 25, 30, 35, 40, 45 or more nucleotides can be employed. However, target sequences of any length can be employed in the methods of the presently disclosed subject matter, and in some cases can be shorter than ten nucleotides and longer than 50 nucleotides. For example, target sequences can be 60 nucleotides long, 75 nucleotides long, 85 nucleotides long, 100 nucleotides long, 300 nucleotides long, or even longer. If desired by the artisan, a target sequence can be fragmented prior to hybridization steps by using enzymatic, mechanical or other means as known in the art.

In certain embodiments, target sequences can be isolated from biological samples, including, but not limited to, bodily fluids (e.g., blood, urine, serum, lymph, saliva, anal and vaginal secretions, perspiration, semen, etc., of virtually any organism); environmental samples (e.g., air, plant, agricultural, water and soil samples); and research samples (i.e., amplification reaction products, purified samples such as purified genomic nucleic acids, and unpurified samples of bacteria, virus, genomic DNA, etc.).

If required, the target nucleic acid can be isolated from source biological samples using known techniques. For example, samples can be collected and concentrated or lysed, as required. Appropriate adjustment of pH, treatment time, lytic conditions and sample modifying reagents can also be made in order to optimize reaction conditions. Such modification techniques are well known to those of skill in the art.

Methods for nucleic acid isolation and purification can comprise simultaneous isolation of, for example, total nucleic acid, or separate and/or sequential isolation of individual nucleic acid types (e.g., genomic DNA, cDNA, organelle DNA, genomic RNA, mRNA, polyA⁺ RNA, rRNA, tRNA) followed by optional combination of multiple nucleic acid types into a single sample.

Methods for nucleic acid isolation can optionally be optimized to promote recovery of pathogen-specific nucleic acids. In some organisms, for example fungi, protozoa, gram-positive bacteria, and acid-fast bacteria, cell lysis and nucleic acid release can be difficult to achieve using general procedures, and therefore a method can be chosen that creates minimal loss of the pathogen subset of the sample.

Semi-automated and automated extraction methods can also be used for nucleic acid isolation, including for example, the SPLIT SECOND™ system (Boehringer Mannheim of Indianapolis, Ind., United States of America), the TRIZOL™ Reagent system (Life Technologies of Gaithersburg, Md., United States of America), and the FASTPREP™ system (Bio 101 of La Jolla, Calif., United States of America). See also Smith (1998) The Scientist 12(14):21-24 and Paladichuk (1999) The Scientist 13(16):20-23.

In some embodiments, a target nucleic acid comprises a double-stranded nucleic acid. Double stranded nucleic acid sequences can be prepared, for example, by isolating a double stranded segment of DNA.

Alternatively or in addition, multiple copies of single stranded complementary oligonucleotides can be synthesized and annealed to one other under appropriate conditions. In order to provide a single-stranded target for hybridization, double-stranded nucleic acids are preferably denatured before hybridization. The term “denaturing” refers to the process by which strands of oligonucleotide duplexes are no longer base-paired by hydrogen bonding and are separated into single-stranded molecules. Methods of denaturation are well known to those skilled in the art and include thermal denaturation and alkaline denaturation.

RNA isolation methods are known to one of skill in the art. See, Albert et al. (1992) J. Virol. 66:5627-2630; Busch et al. (1992) Transfusion 32:420-425; Hamel et al. (1995) J. Clin. Microbiol. 33:287-291; Herrewegh et al. (1995) J. Clin. Microbiol. 33:684-689; Izraeli et al. (1991) Nuc. Acids Res. 19:6051; McCaustland et al. (1991) J. Virol. Methods 35:331-342; Natarajan et al. (1994) PCR Methods Appl 3:346-350; Rupp et al. (1988) BioTechniques 6:56-60; Tanaka et al. (1994) J. Gen. Virol. 75:2691-2698; and Vankerckhoven et al. (1994) J. Clin. Microbiol. 30:750-753.

When mRNA is selected as a target sequence, the methods described herein can enable an assessment of pathogen gene expression. For example, detecting a pathogen in a biological sample can comprise determination of expressed virulence factors, other deleterious agents produced by a pathogen, or biosynthetic enzymes that generate virulence or other harmful pathogen gene products. Such analysis can facilitate distinction between active and latent infection, and indicate severity of an infection.

One of the advantages of the sandwich assay embodiments described herein is that the need to use nucleic acid amplification technology, cell culture, or other methods of selectively amplifying a target nucleic acid sequence are greatly reduced or eliminated. However, while amplification steps are generally not required, procedures that include amplification prior to carrying out the detection methods of the presently disclosed subject matter can be desirable in some cases. Nucleic acid “amplification” generally includes methods such as polymerase chain reaction (PCR), ligation amplification (or ligase chain reaction, LCR) and amplification methods based on the use of the enzyme Q-beta replicase. These methods are well known and widely practiced in the art, and reagents and apparatus for conducting them are commercially available.

Other amplification techniques are known in the art and can be used in conjunction with the detection methods described herein. These methods include random-primed PCR (RP-PCR); linker/adaptor-based DNA amplification; sequence-independent, single-primer amplification (SISPA); whole genome PCR; primer-extension pre-amplification (PEP); transcription-based amplification (variously called self-sustaining sequence replication, nucleic acid sequence-based amplification (NASBA), or transcription-mediated amplification (TMA)), amplified antisense RNA (aRNA); global RNA amplification, and others. See, e.g., Kinzler & Vogelstein (1989) Nuc Acids Res 17(10):3645-3653; Peng et al. (1994) J. Clin. Pathol. 47:605-608); Reyes & Kim (1991) Mol. Cell Probes 5:473-481; Van Gelder et al. (1990) Proc Natl Acad Sci USA 87:1663-1667; Wang et al. (2000) Nat. Biotech. 18(4):457-459; Podzorski et al. in Murray et al., eds., Manual of Clinical Microbiology (American Society for Microbiology, Washington, D.C. (1995) p.130); Zhang et al. (1992) Proc. Natl. Acad. Sci. USA 89:5847-5851; and U.S. Pat. No. 6,066,457 to Hampson et al.

In accordance with the methods described herein, any one of the above-mentioned amplification methods or related techniques can be employed to amplify the target nucleic acid sample and/or target sequence, if desired. In addition, such methods can be optimized for amplification of a particular subset of nucleic acid (e.g., genomic DNA versus RNA), and representative optimization criteria and related guidance can be found in the art. See, e.g., Cha & Thilly (1993) PCR Methods Appl. 3:S18-S29; Linz et al. (1990) J. Clin. Chem. Clin. Biochem. 28:5-13; Robertson & Walsh-Weller (1998) Methods Mol. Biol. 98:121-154; Roux (1995) PCR Methods Appl. 4:S185-S194; Williams (1989) BioTechniques 7:762-769; and McPherson et al., PCR 2: A Practical Approach (IRL Press, New York, N.Y. (1995)).

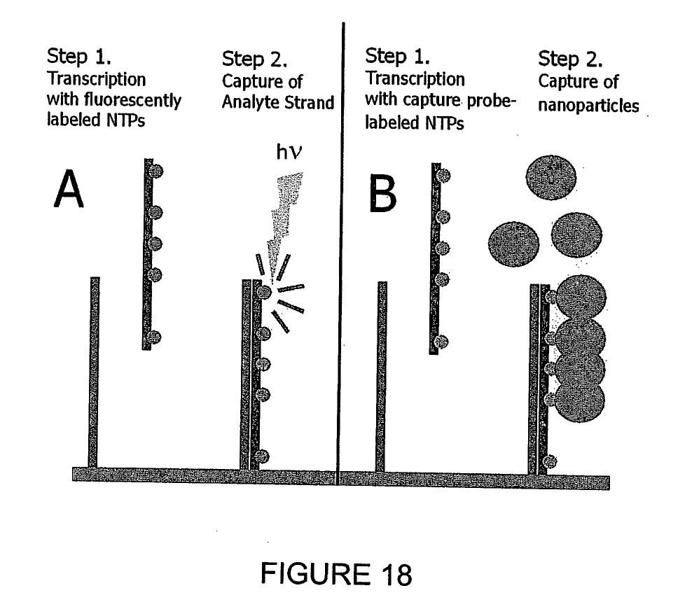

In some embodiments, amplification techniques are used to incorporate labeling or tagging moieties into a target sequence, which moieties are used to facilitate binding to a detection probe. In some embodiments, a target nucleic acid comprises a nucleic acid labeled or tagged with one partner of the ligand-binding pair (e.g., biotin), while a detection probe comprises a nanoparticle attached to the other partner of the ligand-binding pair (e.g., streptavidin). FIGS. 18A and 18B illustrate one method by which a labeling moiety such as biotin can be incorporated into a target sequence. FIG. 18A schematically illustrates a known method of incorporating a fluorescent label into a target nucleic acid, in which a target is amplified using fluorescently-labeled nucleotide triphosphates (NTPs). In some embodiments of such a method, a target sequence is, for example, mRNA, and the complement of the target is enzymatically synthesized by means of a reverse transcriptase to produce a fluorescently-labeled cDNA target strand. Upon binding (hybridization) of a detection probe, the hybridization complex is exposed to light and detected by fluorescent detection and imaging means. FIG. 18B illustrates a method useful in the practice of the present methods, by which biotin-labeled (rather than fluorescently-labeled) NTPs are incorporated into a cDNA target strand, and then used to hybridize nanoparticles coated with streptavidin. Methods of incorporating label and tag moieties (e.g., fluorescent labels, biotin, etc.) into target sequences using transcriptase-based amplification and other methods are known in the art. See, e.g., U.S. Pat. Nos. 6,589,737; 6,046,038; 6,004,755; 6,203,989; 6,589,742 and 6,503,711.

Thus, in some embodiments, a target sequence is labeled with biotin during an amplification reaction in which RNA is used as a template, and nucleotides labeled with biotin are incorporated into a complementary cDNA strand using reverse transcriptase.

2. Probes

The term “probe,” as used herein, indicates a structure, complex or molecule that is able to selectively or substantially hybridize or otherwise bind a target sequence present in a heterogeneous mixture of nucleic acid molecules. In some embodiments, probes comprise oligonucleotide molecules. Oligonucleotide probes are typically designed to hybridize to target sequences in order to determine the presence or absence of the target sequence in a sample. As such, oligonucleotide probes as used in the methods described herein are generally designed to be complementary, in whole or in part, to a target sequence, such that hybridization between the target sequence and the probe or probes occurs.

The term “complementary sequences”, as used herein, indicates two nucleotide sequences that comprise antiparallel nucleotide sequences capable of pairing with one another upon formation of hydrogen bonds between base pairs. Additionally, the term “complementary sequences” means nucleotide sequences that are substantially complementary, as can be assessed by hybridization to the nucleic acid segment in question under relatively stringent conditions such as those described herein. The term “complementary sequence” also includes a pair of nucleotides that bind a same target nucleic acid and participate in the formation of a triplex structure as described, for example in U.S. Pat. No. 6,027,893 to Ørum et al. This complementarity need not be perfect; there can be any number of base pair mismatches which will interfere with hybridization between the target sequence and the single stranded nucleic acids of the presently disclosed subject matter. However, if the number of mutations is so great that no hybridization can occur under even the least stringent of hybridization conditions, the sequence is not a complementary target sequence.

In some embodiments, each probe comprises at least one oligonucleotide sequence, which is complementary to a contiguous nucleic acid sequence of a target sequence such that the oligonucleotide sequence specifically hybridizes to the target sequence under stringent conditions.

The total length of a probe oligonucleotide will vary depending on its use, the length of the target sequence, and the hybridization and wash conditions. In general, oligonucleotide sequences of 5 to 50 nucleotides can be used; however, shorter or longer sequences can, in certain instances, be employed. In some cases, longer probes can be used, e.g., from about 50 to about 200-300 nucleotides or even longer in length.

In some embodiments, single-stranded DNA is used as an oligonucleotide component of the probes used in the present methods. In some embodiments, two oligonucleotides complementary to separate, non-overlapping segments, regions or domain of a target nucleic acid sequence are used in the sandwich hybridization format. In this embodiment, one of the oligonucleotides is used as a capture probe, while the other comprises the oligonucleotide component of the corresponding detection probe. By using two non-overlapping, non-complementary probes to identify a target nucleic acid sequence, the risk of “background noise” being interpreted as a false positive reading is reduced as compared to a system that relies on the hybridization of a single probe for detection.

Methods of making oligonucleotides of a predetermined sequence are well-known. See, e.g., Sambrook et al., supra, and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides. Oligoribonucleotides and oligodeoxyribonucleotides can also be prepared enzymatically.

C. Electrode Materials

Electrodes useful in the present methods facilitate strong, quantifiable electric current when a hybridization complex comprising a capture probe, its complementary target sequence, and a detection probe comprising a nanoparticle is formed, and the nanoparticle is photoexcited in the presence of a redox mediator. At the molecular level, the attachment of the hybridization complex to the electrode positions the nanoparticle in close proximity to the electrode surface, such that an electrical current is generated when an electron is transferred from the nanoparticle to the electrode after light induction.

As used herein, the term “electrode” means a composition which, when connected to an electronic detection device, is able to carry or sense a current or charge, and then convert it to a measurable signal. In some embodiments, an electrode is a solid substrate comprising conducting material.

Suitable electrode material can be selected according to desired redox potential range, ease of surface attachment of nucleic acid to surface, and correct or desired optical properties. As provided above, one limitation in the selection of an electrode material is that it cannot be identical to the material that the detection probe nanoparticle comprises. Electrode materials include, but are not limited to, certain metals and their oxides, such as gold, platinum, palladium, aluminum, indium tin oxide (ITO), tin oxide, fluorine-doped tin oxide, cadmium oxide, iridium oxide, ruthenium oxide, zinc tin oxide, antimony tin oxide; platinum oxide, titanium oxide, palladium oxide, aluminum oxide, molybdenum oxide, tungsten oxide, and others. In some embodiments, the electrode comprises indium tin oxide (ITO). Representative electrode materials are described in Brewer et al., (2002) J. Phys. Chem. B 106:11446.

The electrode can comprise a single conductive material or multiple conductive materials. The conductive electrode material can be layered over a second material, such as a polymer or otherwise non-conducting surface. In some embodiments, the electrode is formed on a solid, non-conducting substrate. The substrate can comprise a wide variety of materials, including but not limited to glass, fiberglass, teflon, ceramics, silicon, mica, plastic (including acrylics, polystyrene and copolymers of styrene and other materials, polypropylene, polyethylene, polybutylene, polycarbonate, polyurethanes, TEFLON™, combinations thereof, and the like. Alternatively or in addition, a support can be constructed from a polymer material such as high density polyethylene (HDPE) used in 96-well titer plates. In yet another example, a polyacrylamide gel can be employed as a solid support for the electrode (Dubiley et al., (1997) Nucleic Acids Res. 25: 2259-2265).

Solid substrates on which electrodes can be formed also include printed circuit board materials. Circuit board materials are those that comprise an insulating substrate that is coated with a conducting layer and processed using lithography techniques, particularly photolithography techniques, to form the patterns of electrodes and interconnects (sometimes referred to in the art as interconnections or leads). The insulating substrate is generally, but not always, a polymer. As is known in the art, one or a plurality of layers can be used, to make either “two dimensional” (e.g., all electrodes and interconnections in a plane) or “three dimensional” (wherein the electrodes are on one surface and the interconnects can go through the board to the other side) boards. Three dimensional systems frequently rely on the use of drilling or etching, followed by electroplating with a metal such as copper, such that the “through board” interconnections are made. Circuit board materials are often provided with a foil already attached to the substrate, such as a copper foil, with additional copper added as needed (for example for interconnections), for example by electroplating. The copper surface may then need to be roughened, for example through etching, to allow attachment of the adhesion layer.

The electrodes described herein are depicted in the Figures as a flat surface. However, a flat surface is only one of the possible conformations of the electrode, and as such is illustrated for schematic purposes only. The conformation of the electrode will vary with the detection method used. For example, flat planar electrodes may be preferred for methods requiring addressable locations for detection.

In some embodiments, and as discussed in more detail herein, the electrode can optionally and further comprise a passivation agent. As used herein, the term “passivation” generally means the alteration of a reactive surface to a less reactive state. Passivation can refer to, for example, decreasing the chemical reactivity of a surface or to decreasing the affinity of a surface for nucleic acids. Stated differently, passivation is a method by which a surface is coated with a moiety having the ability to block subsequent binding to the surface at points where the moiety is bound.

In some embodiments, a passivation agent is in the form of a monolayer on the electrode surface. The efficiency of hybridization can increase when the detection probe is at a distance from the electrode. A passivation agent layer facilitates the maintenance of the probe away from the electrode surface. In addition, a passivation agent can serve to keep charge carriers away from the surface of the electrode. Thus, this layer can help to prevent direct physical or electrical contact between the electrodes and the nanoparticles of the detection probes, or between the electrode and charged species within the redox compound solution. Such contact can result in a direct “short circuit” or an indirect short circuit via charged species which can be present in the sample. Accordingly, the monolayer of passivation agents is preferably tightly packed in a uniform layer on the electrode surface, such that a minimum of “holes” exist.

D. Modification of Electrode Surface with Capture Probes

In some embodiments, the electrode comprises a plurality of capture probes attached to the electrode in an array format. As used herein, the terms “nucleic acid microarray,” and “nucleic acid hybridization array” are used interchangeably, and mean an arrangement of a plurality of nucleic acid sequences (e.g., capture probes) bound to a support (e.g., an electrode). The terms “addressable array” and “array” are used interchangeably, and mean a plurality of entities arranged on a support in a manner such that each entity occupies a unique and identifiable position. In the methods described herein, the entities are capture probes (e.g., capture oligonucleotides) immobilized to the surface of an electrode. As used herein, the terms “immobilize” and “attach” are used interchangeably to mean a chemical and/or mechanical association of one moiety with one or more surfaces (e.g., solid surfaces). The association can be covalent or non-covalent, and can be direct or indirect.

In some embodiments, capture probes attached to the surface of an electrode are ordered such that each capture probe sample has a unique, identifiable location on the support. The physical location on the electrode where a capture probe is attached or immobilized is referred to herein as an “attachment point.” The identity of a capture probe bound to an electrode at a given location can be determined in several ways. One exemplary way to correlate a capture probe with its location is to attach the capture probe to the support at a known position (see, e.g., Pirrung, (1997) Chem. Rev. 97: 473-486). Discrete locations on the support can be identified using a grid coordinate-like system. In this approach, the working area of the support surface can be divided into discrete areas that can be referred to interchangeably as “spots” or “patches”. Different capture probes can subsequently be attached to the surface in an orderly fashion, for example, one capture probe, or one sample of identical capture probes, to a spot. In this strategy, the probe oligomers can be applied one or several at a time. In one exemplary method, sites at which it might be desirable to temporarily block probe binding can be blocked with a blocking agent. The blocking agent can be subsequently removed and the site freed for probe binding. This process can be repeated any number of times, thus facilitating the attachment of a known probe at a known location on a support.

Localizing capture probes to an electrode surface at known locations can also involve the use of microspotting. In this approach, the location of the capture probes on an electrode surface is determined by the ordered application of probe samples in a group. That is, capture probes are ordered in known locations prior to application to the electrode surface. In this way, the location of each probe is known as it is applied. Appropriate devices for carrying out this approach are commercially available and can be used with the detection methods described herein. For example, the present methods are compatible with the commercially available GENECHIP™ system (Affymetrix, Inc., Santa Clara, Calif.) or the commercially available SPOTBOT™ Automated Spotting Arrayer (TeleChem International, Sunnyvale, Calif.).

As set forth above, in some embodiments a single-stranded nucleic acid sequence is used as a capture probe. For example, a capture probe can comprise a single-stranded cDNA sequence complementary to a target gene of interest or to a target domain thereof. The capture probe can be attached to the electrode surface indirectly via an “attachment linker,” as defined herein. In this embodiment, one end of an attachment linker is attached to a capture probe, while the other end (although, as will be appreciated by those in the art, it need not be the exact terminus for either) is attached to the electrode.

The method of attachment of the capture probe to the attachment linker can generally be done as known in the art, and will depend on the composition of the attachment linker and the capture probe. In general, the capture probe is attached to the attachment linker through the use of functional groups on each moiety that can then be used for attachment. Preferred functional groups for attachment are amino groups, carboxy groups, oxo groups and thiol groups. Using these functional groups, the capture probes can be attached using complementary functional groups on the electrode surface.

In one example of an attachment approach suitable for attachment of capture probes to an electrode surface, one or more probe capture sequences are initially incubated with a solution of a thio-alcohol for a pre-selected period of time. In some embodiments, C6 mercaptohexanol is employed as a thio-alcohol, in accordance with techniques described by Loweth et al., (1999) Angew. Chem. Int. Edit. 38: 1808-12, and Storhoff & Mirkin, (1999) Chem. Rev. 99: 1849-62. Thio-alcohol and capture probe are added in amounts so as to bring the final concentration of capture probe in the solution to about 20% or less. The incubation time permits the covalent association of the 3′ end of the capture probe oligonucleotide with the hydroxyl group of the thio-alcohol. The solution is then exposed to the surface of a support under conditions that permit association of the sulfur atom of the thio group with the surface of the support. Suitable equipment is commercially available and can be used to assist in the binding of a target sequence to a support surface.

In another specific example, a monolayer of 12-phosphonododecanoic acid is formed on the electrode surface. The carboxylic acid of 12-phosphonododecanoic acid is then activated by 1-ethyl-3-(3-dimethylaminopropyl)carbodiimide hydrochloride (EDC) to form an O-acylisourea intermediate. See, e.g., S. H. Brewer et al., Langmuir (2002) 18, 6857-6865; B. L. Frey and R. M. Corn, Analytical Chemistry (1996) 68, 3187-3193; M. Burgener et al., Bioconjugate Chemistry (2000) 11, 749-754; K. Kerman et al., Analytica Chimica Acta (2002) 462, 39-47; E. Huang et al., Langmuir (2000) 16, 3272-3280; and G. T. Hermanson, Bioconjugate Techniques (1996) (Academic Press: San Diego). This activated carboxylic acid group is attacked by the primary amine (acting as a nucleophile) of a 5′-modified C₃NH₂ single-stranded DNA strand to form an amide bond between the monolayer of 12-phosphonododecanoic acid and the 5′ modified C₃NH₂ ssDNA.

Other functional groups useful for attaching oligonucleotides to solid surfaces (i.e., electrodes and nanoparticles) include, for example, moieties comprising thiols, carboxylates, hydroxyls, amines, hydrazines, esters, amides, halides, vinyl groups, vinyl carboxylates, phosphates, silicon-containing organic compounds, and their derivatives. Still other functional groups useful for attachment include phosphorothioate groups (see, e.g., U.S. Pat. No. 5,472,881 for the binding of oligonucleotide-phosphorothioates to gold surfaces), aminosilanes (see, e.g,. K. C. Grabar et al., J. Am. Chem. Soc. (1996) 118, 1148), and substituted alkylsiloxanes (see, e.g., Burwell, Chemical Technology 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes). Oligonucleotides terminated with a 5′ thionucleoside or a 3′ thionucleoside can also be used for attaching oligonucleotides to solid electrode surfaces. The length of these attaching functional groups is chosen such that the conductivity of these molecules does not hinder electron transfer from the nanoparticle to the electrode via the hybridized probe and target nucleic acids. Stated differently, these functional groups are preferred to have higher conductivities than double-stranded nucleic acid.

In yet another embodiment, a “tag” or “linker” nucleic acid sequence can be employed to attach capture probes to electrode surfaces. When a tag sequence is employed, an electrode can comprise a tag nucleic acid complement. A tag complement is a sequence that is complementary to a tag sequence associated with a capture probe. Thus, when a capture probe comprising a tag sequence is contacted with an electrode comprising a tag complement under suitable hybridization conditions, a duplex can form.

A tag sequence can comprise, for example, a sequence that is complementary to a support-bound tag complement. A tag sequence can be associated with a target sequence, which can then be amplified by PCR prior to association with a nanoparticle. The PCR amplicon will comprise a nucleic acid sequence comprising the tag sequence and a target sequence. The PCR amplicon then comprises a sequence that is complementary to a support-bound tag complement. Inclusion of a tag sequence, for example as a component of a target sequence, offers the advantage that a support need not be specific for a given target sequence, but rather can be universal in the sense that it is specific for a tag complement, but not for any particular target sequence. Thus, by employing a tag complement, an electrode (or nanoparticle, as described herein) can be independent of the source of a capture probe oligonucleotide (as to species, etc.) in the sense that the electrode can be specific for a tag sequence, but not for any particular capture probe sequence. Thus, by employing a method comprising the use of a tag-tag complement approach, the need to form different electrode supports for different probe and/or target sequences is mitigated. See, e.g., WO 94/21820, WO 97/31256, WO 96/41011 and U.S. Pat. No. 5,503,980.

Following attachment of a capture probe to the surface of the electrode, the areas of the electrode surface to which no probe is bound can be passivated, as defined above. A passivation process can be implemented after probes are bound to the support, and can include sequential synthesis and co-deposition approaches, as is known in the art.

In some embodiments, passivation is accomplished by exposing the surface to thio-alcohol, as described above. For example, the same thio-alcohol can be used to passivate the surface as was used in attaching the probe to the surface. In other embodiments, thio-alcohols of shorter or longer length than those used to attach capture probes can be employed.

In another embodiment, other molecules, i.e., “passivation moieties” can be used passivate the surface of a support. For example, polyethylene glycol (PEG), various alcohols and carboxylates can all be used to passivate the surface of a support, as can COO— and CONH₂ moieties. In some embodiments, passivation moieties can also be non-covalently or covalently attached. Indeed, virtually any material can be used to passivate a support surface, with the understanding that the passivation material must associate with the support to form a protective layer coating the support, and that the passivating process, which can be performed after a probe is already associated with the surface of the support, does not damage any probes already bound to the support. As described above, a passivation step can also be performed to reduce the potential for nonspecific association between a nanoparticle complex and a support.

E. Detection Probe Components

Detection probes used in the practice of the presently described methods generally comprise at least two components. In some embodiments, the two components include an oligonucleotide nucleic acid sequence, and a nanoparticle to which the oligonucleotide is attached.

In some embodiments, a non-nucleic acid ligand takes the place of an oligonucleotide sequence. In this embodiment, the non-nucleic acid is a member of a ligand binding pair, and the other member of the binding pair is attached to or is comprised by the target sequence, such that the target sequence can specifically or selectively bind the detection probe. In one example of these embodiments, a target sequence is biotinylated according to methods described above, while a detection probe comprises a nanoparticle coated with streptavidin. Methods for attaching streptavidin to nanoparticles are known, see, e.g., Shaiu et al., Nuc. Acids Res. 21, 99 (1993).

Detection probes can also comprise other useful moieties, including electrochemically-active redox reaction mediators, catalysts, supplementary labeling molecules or detection enhancers, and the like.

As used herein, the terms “nano”, “nanoscopic”, “nanometer-sized”, “nanostructured”, “nanoscale”, and grammatical derivatives thereof are used synonymously, and in some cases interchangeably. As used herein, the term “nanoparticle” can mean any structure comprising a nanoparticle. Typically, but not necessarily, a nanoparticle is an approximately spherical metal atom-comprising entity. In one example, a nanoparticle is a particle comprising a material such as a metal, a metal oxide or a semiconductor. In other examples, a nanoparticle can comprise a polymeric species or any other conducting material.

Nanoparticles are generally less than about 1000 nanometers (nm) in diameter, usually less than about 200 nanometers in diameter and more usually less than about 100 nanometers in diameter. In certain particular embodiments, nanoparticles are between about 10 nm and 20 nm in diameter, while in other embodiments, the size of the nanoparticle is less than about 10 nm. Representative ranges of nanoparticle sizes include but are not limited to from about 5 to about 200 nanometers, from about 5 to about 100 nanometers, from about 5 to about 50 nanometers, from about 5 to 20 nanometers, from about 10 to about 200 nanometers, from about 10 to about 100 nanometers, and from about 10 to about 50 nanometers.

A nanoparticle can comprise almost any material, as long as the material is (1) is different from the electrode material used in the hybridization reactions, and (2) exhibits surface plasmon resonance. The skilled artisan will be able to readily determine whether a putative nanoparticle material exhibits surface plasmon resonance, either because this characteristic of the material is known in the art, or because it can be determined by methods known in the art. See, e.g., B. Liedberg et al., Biosens. Bioelectron. (1995) 10: i-ix, and J. Homola et al., Sensors and Actuators B (1999) 54: 3-15.

In the practice of the methods described herein, materials that can be used in nanoparticle fabrications are able to catalyze electrochemical reactions, and/or are able to alter the rate of electron transfer at an electrode. As such, one consideration when selecting a material for a nanoparticle is the chemical reactivity profile of the material. The chemical reactivity profile of a material is a consideration because other entities, such as oligonucleotides, will ultimately be associated with the nanoparticle. Additionally, it can be desirable to associate an additional, secondary component (e.g., another electrochemically active moiety) with a nanoparticle. Therefore, the reactivity of a nanoparticle to a desired secondary component can also be a consideration. Thus, considerations when selecting and/or designing a nanoparticle can include size, material, chemical reactivity of the material the ease with which an oligonucleotides can associate with the nanoparticle, and the ease with which a secondary component can associate with the nanoparticle.

Nanoparticles can be formed from metals and metal oxides, including but not limited to gold, silver, titanium, titanium dioxide, tin, tin oxide, iron, iron^{III} oxide, copper, nickel, aluminum, steel, indium, platinum, indium tin oxide, fluoride-doped tin, ruthenium oxide, germanium cadmium selenide, cadmium sulfide and titanium alloy. Nanoparticles can also be formed from semiconductor materials (e.g., CdSe, CdS, and CdS or CdSe coated with ZnS) and magnetic (e.g., ferromagnetite) colloidal materials.

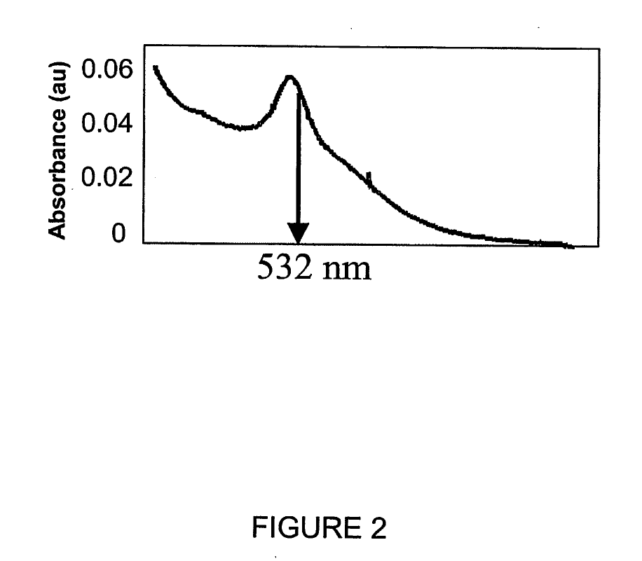

In some embodiments, the nanoparticle material is selected from the group consisting of gold and silver, or combination or alloy of any of the foregoing. In some embodiments, the detection probe nanoparticle comprises gold. See FIG. 2, which illustrates an absorbance spectrum of a gold nanoparticle as a function of increasing light wavelength. When the gold nanoparticle is irradiated at 532 nm (near the surface plasmon resonance of the gold nanoparticle), a jump in absorbance is observed. Similar behavior is exhibited by silver nanoparticles when irradiated with a light wavelength in the 420-460 nm range. As used herein, the term “gold” means element 79, which has the chemical symbol Au, and “silver” means element 47, which has the chemical symbol Ag.

Nanoparticles comprising the above-listed materials are generally available commercially from numerous suppliers, including but not limited to Vacuum Metallurgical Co., Ltd. (Chiba, Japan), Vector Laboratories, Inc. (Burlingame, Calif.), Ted Pella, Inc., Amersham Corporation and Nanoprobes, Inc.

Alternatively or in addition, nanoparticles can be fabricated using a suitable method. See, e.g., Marinakos et al. (1999) Adv. Mater. 11:34; Marinakos et al. (1998) Chem. Mater. 10:1214-19; Enustun & Turkevich (1963) J. Am. Chem. Soc. 85:3317; Hayashi (1987) J. Vac. Sci. Technol. A5(4): 1375-84; Hayashi (1987) Phys. Today, December 1987, 44-60; MRS Bulletin, January 1990, pp. 16-47; G. Schmid, (ed.) Clusters and Colloids (V C H, Weinheim, 1994); M. A. Hayat (ed.) Colloidal Gold: Principles, Methods, and Applications (Academic Press, San Diego, 1991); R. Massart, IEEE Transactions On Magnetics, 17, 1247 (1981); T. S. Ahmadi, et al., Science, 272, 1924 (1996); A. Henglein, et al., J. Phys. Chem., 99, 14129 (1995); A. C. Curtis, et al., Angew. Chem. Int. Ed. Engl., 27, 1530 (1988); Weller, Angew. Chem. Int. Ed. Engl., 32, 41 (1993); Henglein, Top. Curr. Chem., 143, 113 (1988); Henglein, Chem. Rev., 89, 1861 (1989); Brus. Appl. Phys. A., 53, 465 (1991); Bahncmann, in Photochemical Conversion and Storage of Solar Energy (eds. Pelizetti and Schiavello, 1991), page 251, and others.

Special metal-coated particles known as “nanoshells” are also included in the definition of the term “nanoparticles,” in the practice of the present methods. In general, nanoshells comprise a non-conducting, semiconductor or dielectric core coated with an ultrathin metallic layer. In general, nanoshells have diameters ranging from a few nanometers up to about 5 microns, and have defined wavelength absorbance maxima across the visible and infrared range of the electromagnetic spectrum. Gold nanoshells are one class of optically active nanoparticles that consist of a thin layer of gold surrounding a dielectric core, such as gold sulfide (see, e.g., R. D. Averitt et al., J. Opt. Soc. Am. B 16:1824-1832 (1999), and R. D. Averitt et al., Phys. Rev. Left. 78:4217-4220 (1997)), or other materials.

Metal nanoshells possess optical properties similar to metal colloids, i.e., a strong optical absorption and an extremely large and fast third-order nonlinear optical (NLO) polarizability associated with their plasmon resonance. The plasmon resonance frequency of metal nanoshells depends on the relative size of the nanoparticle core and the thickness of the metallic shell (see, e.g., A. E. Neeves, et al. J. Opt Soc. Am. B 6:787 (1989) and U. Kreibig, et al., Optical Properties of Metal Clusters (Springer, N.Y. (1995)). By adjusting the relative core and shell thickness, metal nanoshells can be fabricated that will absorb or scatter light at any wavelength across the entire visible and infrared range of the electromagnetic spectrum. The relative size or depth of the particle's constituent layers determines the wavelength of its absorption. Whether the particle absorbs or scatters incident radiation depends on the ratio of the particle diameter to the wavelength of the incident light.

For any given core and shell materials, the maximum absorbance of the particle depends upon the ratio of the thickness (i.e., radius) of the core to the thickness of the shell. Based on the core radius:shell thickness (core:shell) ratios that are achieved by the referenced synthesis method, nanoshells manifesting plasmon resonances extending from the visible region to approximately 5 μm in the infrared can be readily fabricated. By varying the conditions of the metal deposition reaction, the ratio of the thickness of the metal shell to the core radius can be varied in a predictable and controlled way. Accordingly, particles are constructed with core radius to shell thick ratios ranging from about 2-1000. This large ratio range coupled with control over the core size results in a particle that has a large, frequency-agile absorbance over most of the visible and infrared regions of the spectrum.

The nonlinear optical (NLO) properties of metal nanoshells or nanoshells-constituent materials can be resonantly enhanced by judicious placement of the plasmon resonance at or near the optical wavelengths of interest. The extremely agile “tunability” of the plasmon resonance is a property particular to metal nanoshells. The resonance of the optical absorption and NLO properties of a nanoshell can thus be systematically designed.

F. Attachment of Binding Partners to Nanoparticles

The alkanethiol method described above in reference to attaching oligonucleotides to electrode surfaces can also be used to attach oligonucleotides to nanoparticle components of detection probes. For instance, oligonucleotides functionalized with alkanethiols at their 3′-termini or 5′-termini readily attach to gold nanoparticles. See, e.g., Whitesides, Proceedings of the Robert A. Welch Foundation 39th Conference On Chemical Research Nanophase Chemistry, Houston, Tex., pp. 109-121 (1995); Mucic et al., Chem. Commun. (1996) 555-557.

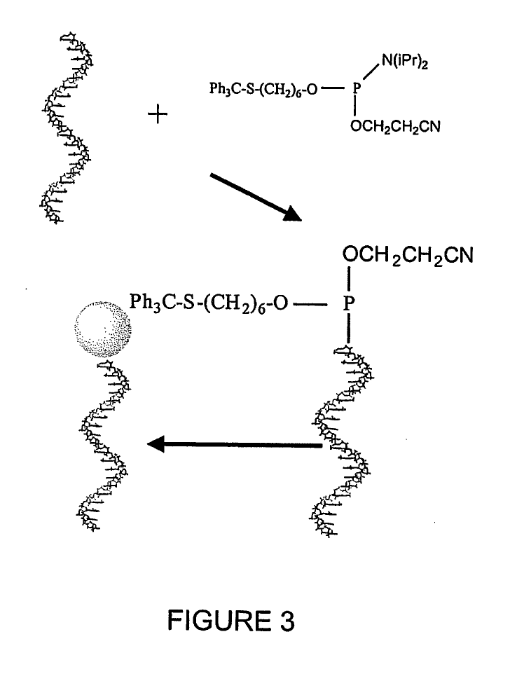

When attaching an oligonucleotide probe to a nanoparticle, a thiolation reaction can be performed to add a thiol group to the 5′ end of a single-stranded oligonucleotide. Alternatively or in addition, an amination reaction can be performed and will proceed mutatis mutandis with the thiolation reaction described herein. The general purpose of the reaction is to introduce a nucleophilic center that can subsequently be functionalized with a nanoparticle as described herein. As shown in FIG. 3 and immediately below, a suitable thiol modifier phosphoramidite reagent is the following compound, which is available from Glen Research, Corp. of Sterling, Va.:

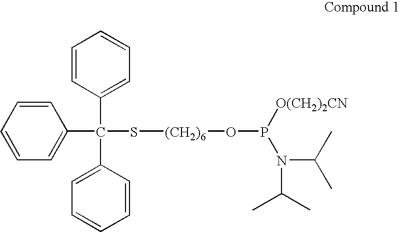

Referring now to FIG. 3, single-stranded oligonucleotides are incubated with a thiol modifier phosphoramidite under anhydrous conditions that permit attachment of the phosphine to the 5′ end of the oligonucleotide. The reaction can be carried out in a nucleic acid synthesizer under standard (and anhydrous) conditions. The thiol modifier is generally added in the last step of synthesis of an oligonucleotide. The phosphine is oxidized using iodine, and the purification is generally the same as that used for unlabeled oligonucleotides. In this reaction, the thiol group is generally protected by a protecting trityl or acetic thioester group and is separated from the 5′-phosphodiester by a variable-length carbon linker. A six-carbon linker is represented in the structure of Compound 1.

The oligonucleotide complex is then subjected to thiol deprotection to remove the trityl group. Specifically, the protecting trityl group is removed by treatment with silver nitrate and dithiothreitol (DTT). The oligonucleotide complex is then incubated with a nanoparticle. The two entities are joined at the thiol exposed by the removal of the trityl group during the deprotection reaction. The formed nanoparticle-oligonucleotide conjugates (i.e., detection probes) can be maintained in the reaction vessel until use.

When a non-synthetic (i.e., isolated, extended or reverse transcribed) oligonucleotide is employed as a component of the detection probe in the presently disclosed subject matter, the oligonucleotide can be attached to a nanoparticle in a variety of ways. One mechanism for attaching a non-synthetic oligonucleotide probe to a nanoparticle, generally described as an “end-labeling” scheme, involves derivatizing the 5′ hydroxyl group of an oligonucleotide to incorporate a functional group reactive with the nanoparticle material on the 5′ end of the oligonucleotide. A representative, but non-limiting, list of functional groups includes carboxylate groups, amine groups and thiols group. Such functional groups can be added to an oligonucleotide as a step in the synthesis of the oligo and can be programmed as an additional step in automated nucleic acid synthesizers, as is known in the art.

In some embodiments of an attachment scheme, an oligonucleotide having a 5′ hydroxyl group is incubated under suitable anhydrous reaction conditions with N,N′ carbonyldiimidazole and subsequently with a cysteamine, thereby end labeling the oligo with a thiol group according to Reaction Scheme 1:

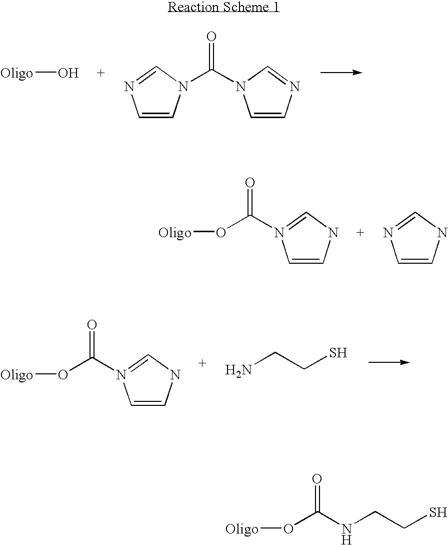

In yet another embodiment of an attachment scheme, a carboxylate (or a thiol, amine or any other moiety) moiety can be chemically incorporated into a reverse transcription reaction or, as noted, attached to the 5′ hydroxyl of a synthesized oligonucleotide. Similarly, phosphonates and amines can be employed to attach an oligonucleotide to a metal oxide or a nanoparticle. Cystamine-based attachment strategies can also be employed. Those of ordinary skill in the art can recognize reaction conditions that might be damaging to an oligonucleotide and can design attachment strategies, using the above disclosure as a guide, so as to maintain the integrity of the oligonucleotide. It is noted that a deoxynucleotide phosphate (dNTP) having a 5′ hydroxyl group can also be derivatized using Reaction Scheme 1 for attachment to a nanoparticle. Suitable protective groups and additional reaction conditions can be employed, and are known to those of skill in the art.

Although the examples provided above illustrate the attachment of one moiety (i.e., an oligonucleotide) to one nanoparticle, the present methods specifically encompass embodiments in which a plurality of moieties is attached to a single nanoparticle (i.e., the nanoparticles of the present methods are polyvalent). In some embodiments, a plurality of identical oligonucleotides is attached to one nanoparticle. In another embodiment, one or more identical oligonucleotide sequences are attached to the nanoparticle, as well as one or more other, non-oligonucleotide embodiments (e.g., one or more electrochemically active moieties, as defined herein).

G. Sandwich Format Hybridization Assays

After a capture probe has been immobilized to an electrode surface, a target nucleic acid has been selected and a detection probe comprising a nanoparticle has been prepared, a series of hybridization reactions are performed in the sandwich assay format. Generally, a target sequence is brought into contact with an electrode whose surface has been modified by attaching capture probes to the electrode surface. The target sequence can be brought into contact with the capture probe under hybridization conditions in any suitable manner. In some embodiments, the target sequence is solubilized in a solution, and the target sample is contacted with the capture probe by immersing the electrode having the capture probe immobilized thereon into the solution containing the target sample.

If the capture oligonucleotide and the target nucleic acid are complementary sequences, the target sequence will hybridize with the capture probe, thus forming a first hybridization complex comprising a capture probe and a target sequence. After capture and target nucleic acids have been permitted to hybridize, any unbound (unhybridized) nucleic acid can be removed from the surface of the electrode.

In some embodiments, the capture probes attached to the electrode have sequence complementary to a first domain of the target sequence to be detected. The target sequence is contacted with the capture probe under conditions effective to allow hybridization of the capture probe with the target. In this manner, the target becomes bound to the capture probe. Any unbound target sequence can optionally be removed from the electrode before adding a detection probe, as defined herein.

To complete the sandwich assay, the electrode surface (with capture probe-target sequence hybridization complexes attached thereto) is brought into contact under hybridization conditions with a detection probe comprising a nanoparticle. If a first hybridization complex has formed at a location on the electrode, the detection probe will bind or hybridize the target sequence component of the first hybridization complex, thus forming a second hybridization complex comprising a capture probe, a target sequence and a detection probe comprising a nanoparticle. Thus, the second hybridization complex is attached to the electrode surface by means of the capture probe. The hybridization steps can be performed in any order, or simultaneously, with or without intervening wash steps.

In some embodiments, the detection probe comprises an oligonucleotide component having sequence complementary to a second domain of the target nucleic acid, and the contacting takes place under conditions effective to allow hybridization of the oligonucleotides attached to the nanoparticle to the target sequence. In this manner, detection probe nanoparticles become attached to the electrode as part of a hybridization complex. After the detection probe has been hybridized to the target, unbound nanoparticle-oligonucleotide conjugates and can be removed from the electrode.

Thus, the methods described herein utilize capture and detection probes that substantially hybridize or bind to a target sequence. The phrases “hybridizing substantially to” and “substantially hybridizes” refer to complementary hybridization between a probe nucleic acid molecule and a target nucleic acid molecule, and embraces hybridization of substantially identical sequences that can be accommodated by adjusting the stringency of the hybridization media to achieve the desired hybridization.

The terms “specifically hybridizes” and “selectively hybridizes” each refer to binding, duplexing, or hybridizing of a molecule only or highly preferably to a particular nucleotide sequence when that sequence is present in a complex or heterogeneous nucleic acid mixture.

An extensive guide to the hybridization of nucleic acids is found in Tijssen, Laboratory Techniques in Biochemistry and Molecular Biology-Hybridization with Nucleic Acid Probes (Elsevier, New York, N.Y. (1993) Part I, Chapter 2). A variety of hybridization conditions can be used in the presently disclosed subject matter, including high, moderate and low stringency conditions; see for example Maniatis et al., supra, and Ausubel, et al., supra. Hybridization conditions can also vary when a non-ionic backbone, i.e., PNA is used, or when the detection probe and target sequence comprise complementary partners of a ligand binding pair (e.g., streptavidin and biotin), as is known in the art.

Stringent conditions are those that allow hybridization between two nucleic acid sequences with a high degree of homology, but preclude hybridization of random, non-complementary sequences. In general, hybridization at low temperature and/or high ionic strength is termed low stringency, and hybridization at high temperature and/or low ionic strength is termed high stringency. The temperature and ionic strength of a desired stringency are understood to be applicable to particular lengths of nucleic acid sequences, to the base content of the sequences, and to the presence of other compounds such as formamide in the hybridization mixture.

Stated otherwise, “stringent hybridization conditions” and “stringent hybridization wash conditions,” in the context of nucleic acid hybridization experiments, are both sequence- and environment-dependent. In general, longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

One can employ varying conditions of hybridization to achieve varying degrees of selectivity of probe towards target sequence, with the general rule that the temperature remain within approximately 10° C. of the duplex's predicted Tₘ, which is the temperature (under defined ionic strength and pH) at which 50% of the target sequence hybridizes to a perfectly matched probe. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5M NaCl at 65° C. An example of stringent wash conditions is 15 minutes in 0.2×SSC buffer at 65° C. A high stringency wash can optionally be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C.

For shorter sequences (e.g., about 10 to 50 nucleotides), stringent conditions typically involve incubation in salt concentrations of less than about 1.0 M sodium ion, typically about 0.01 to 1.0 M sodium ion (or other ion) concentration, at pH 7.0-8.3, at a temperature of at least about 30° C.

For applications requiring high selectivity, one will typically desire to employ relatively stringent conditions to form hybridization complexes, e.g., conditions of high stringency where one will select relatively low salt and/or high temperature conditions, such as provided by about 0.02 M to about 0.15 M NaCl at temperatures of about 50° C. to about 70° C. Such selective conditions tolerate little, if any, mismatch between the probe and the target strand.

It is generally appreciated that conditions can be rendered more stringent by the addition of increasing amounts of formamide, which serves to destabilize the hybridization complex in the same manner as increased temperature. Thus, hybridization conditions can be readily manipulated, and conditions can be readily selected depending on the desired results.

H. Electrochemical Reactions

The electrode and any bound hybridization complexes are subsequently or simultaneously exposed to a redox solution comprising a redox mediator. The redox solution, in some embodiments, further comprises an electrolyte (i.e., is an electrolyte solution comprising a redox mediator). In some embodiments, the assembly comprising the electrode and any attached hybridization complexes is immersed into an aqueous, redox solution comprising a redox mediator, and an electrolyte. Alternatively or in addition, the hybridization reactions of target sequences to the capture and detection probes are carried out in the presence of the redox solution. In some embodiments, the hybridization steps and exposure to a redox solution steps occur simultaneously and/or are carried out in the same reaction chamber.

As used herein, the terms “redox mediator” and “redox compound” are used interchangeably and mean a redox-active molecule or part of a molecule that is capable of undergoing changes in its electronic properties. The terms “redox-active moiety” or “redox-active molecule” refers to a compound that can be oxidized and reduced, i.e., which contains one or more chemical functions that accept and transfer electrons.

Redox mediators are thus chemical species capable of being reduced and/or oxidized. Redox mediators include, but are not limited to, metals and metals ions, organic compounds capable of being reduced and/or oxidized, and inorganic compounds capable of being reduced and/or oxidized.

In some embodiments, the redox mediator comprises a redox couple. Redox couples are analytes differing only in oxidation state. By way of example, a redox couple that can be used in the present methods is ferricyanide/ferrocyanide, or Fe(CN)₆³⁻/ Fe(CN)₆⁴⁻. The anion, Fe(CN)₆³, contains an iron atom in the +3 oxidation state. At the surface of an electrode, a single electron can be added to the ferricyanide anion. This causes it to be reduced to the anion, Fe(CN)₆⁴⁻, which contains an iron atom in the +2 oxidation state. Other redox couples include ferriin-ferroin, ferrocene/ferrocinium, EDTA/EDTA⁻¹, H₂O/H₂ redox couple and O₂/H₂O₂.

In some embodiments, the redox mediator is an inorganic redox couple, generally employing an iron or ruthenium couple. The iron can be in any convenient form, and in some embodiments is coordinated, such as with hexacyanoferrate, ferricyanide/ferrocyanide, ferriin-ferroin, ferrocene/ferrocinium, or other stable form of iron that is capable of undergoing one-electron transfer.

In some embodiments, the redox mediator is a metallocene or a derivative thereof. In some embodiments, the redox mediator is a ferrocene (such as ferrocene itself), or a derivative thereof. A ferrocene has, as its fundamental structure, an iron atom held “sandwiched” by dative bonds between two pentadienyl rings. It is an electroactive organometallic compound, acting as a pH-independent reversible one-electron donor. The electrochemistry of ferrocene has been characterized. See, e.g., Uosaki et al., (1991) Langmuir7: 1510; Chidsey et al., (1990) J. Am. Chem. Soc. 112: 4301; Tender et al., (1994) Anal. Chem. 66: 3173.

Suitable redox mediators thus include ferrocene and its derivatives, which include 1,1′-ferrocene dicarboxylic acid, 1,1′-dimethylferrocene (DMF), polyvinylferrocene (having monomeric ferrocene or a monomeric ferrocene derivatives such as (ferrocene)₄ and “boron tetraferrocene” or [B(ferrocene)₄)]), [N-ferrocenoyl]-4-aminophenyl phosphate, and ferrocene monocarboxylic acid (FMCA).

Other redox mediators can be selected from the groups including but not limited quinones (e.g., benzoquinone), phenylene diamines, metal complexes with organic ligands tetracyanoquinodimethane, N,N,N′,N′-tetramethyl-p-phenylenediamine, 2,6-dichloroindophenyl phosphate, tetrathiafulvalene, coordinated ruthenium compounds, carboranes, conductive salts of tetracyanoquinodimethane (TCNQ), haloanils and derivatives thereof, vologens, alkyl substituted phenazine derivatives, 3,3′,5,5′-tetramethylbenzidine, bis-cyclo pentadienyl complexes of transition metals; and phenol derivatives including ferrocene-phenol and indophenol compounds.

Preferred redox mediators facilitate slow redox at the electrode surface, and fast redox at the nanoparticle. In some embodiments, the redox mediator is the EDTA/EDTA⁻¹ redox couple. In some embodiments, redox mediator is the H₂O/H₂ redox couple. In still another embodiment, the redox mediator is the O₂/H₂₂ redox couple.

The redox mediator can be present in solution at any appropriate concentration, for example in the range of about 1.0 to about 1000 μM, and from about 10 to about 200 μM, optionally depending on the selection of the mediator.

The nanoparticle itself serves as a “redox-active signal”. That is, a single gold nanoparticle comprises tens of thousands of gold atoms that can be oxidized to Au³⁺ ions. This oxidation reaction can be detected electrochemically. This approach offers the advantage that the signal amplification factor is very large.

Electrochemical contact is advantageously provided using an electrolyte solution in contact with each of the electrodes or microelectrode arrays of the presently disclosed subject matter. The medium must be conducting. This can be achieved by using an electrolyte solution. An electrolyte solution is made by adding an ionic salt to an appropriate solvent.

Selection of the appropriate electrolyte for the redox solution can be made according to known parameters. Electrolyte solutions that can be used in the apparatus and methods of the presently disclosed subject matter include any electrolyte solution at physiologically-relevant ionic strength (equivalent to about 0.15M NaCl) and neutral pH (e.g., pH 7.0 to 7.6). The salt must become fully dissociated in the solvent in order to generate a conducting (i.e., ionic) solution.

Electrolyte solutions can be aqueous or non-aqueous. A wide range of salts can be used for aqueous electrolyte solutions. Since the redox potentials of some compounds are pH sensitive, buffered solutions should be used for these compounds. Solvents suitable for non-aqueous solutions include, but are not limited to, acetonitrile, DMF, DMSO, THF, methylene chloride, and propylene carbonate.

Various buffers can be employed in the electrolyte solution, which include but are not limited to tris-(hydroxymethyl) methylamine (Tris), phosphate, borate, or the like, usually employing a buffer suitable for the particular enzyme system. Non-limiting examples of electrolyte solutions useful with the methods described herein include, but are not limited to, saline, phosphate buffered saline, potassium nitrate, HEPES buffered solutions, and sodium bicarbonate buffered solutions.

I. Detection of Electrochemical Reaction

As used herein, the term “detect” means determining the presence of a target molecule, entity or event. Determination is carried out by observing the occurrence of a detectable signal (e.g., an electrical, chemical, visual or spectroscopic signal) that occurs in the presence of the target molecule or entity, or during the occurrence of the target event (i.e., a hybridization event).

As used herein, the term “electrical current” means the movement of electrons from a higher energy level to a lower energy level. Generally, electrical current is the flow of electrical charge, and the term can also refer to the rate of charge flow through a circuit.

After formation of hybridization complexes, and while in contact with the redox solution, the electrode surface is exposed to light, as explained in more detail below. The light catalyzes an electrochemical reaction, whereby an electrical charge is transferred from the redox mediator in solution to the electrode surface via the hybridization complex. Catalytic current or electrochemical signal is not generated in significant amounts by non-hybridized capture probes, because these capture probes are not also attached to a detection probe comprising a nanoparticle. The electron transfer generates a current in the electrode, which can be detected. In some embodiments, the generated electrical current is measured for each nucleic acid-modified electrode, and compared to a reference current obtained with the complex-free electrode. In another embodiment, a comparison between the electrode potential of the complex-free electrode and the potential of the irradiated, modified electrode is made.

As used herein, when referring to a compound, the term “electroactive” means the compound has the ability to change electronic configuration. The term refers to a molecule or structure and includes the ability to transfer electrons, the ability to act as a conductor of electrons and the ability to act as an electron donor or acceptor. The term specifically encompasses the ability of a molecule to act as the donor in an electron transfer when it is excited by light (i.e., is “photoexcited”).

As used herein, the term “photoelectrochemically active”, and grammatical derivations thereof, means having the ability to transfer or transport electrons following photoexcitation by light. Generally, the term refers to a chemical entity that can be promoted to an excited state by absorption of energy at a given wavelength and can act as an electron donor or acceptor.

As used herein, the term “photoelectrochemically active moiety” means any structure adapted to generate or carry an electric current generated in response to the application of light. For example, a photoelectrochemically active moiety can comprise a structure comprising a photoinducible electron donor, which can act as a donor in a photoinduced electron transfer reaction; as a photoredox agent, which can act as the acceptor in a photoinduced electron transfer reaction; or as a sensitizer or mediator, which can act in a manner analogous to the role of a catalyst in a chemical reaction.

In some embodiments of the present methods, an additional electrochemically active moiety is added to the redox electrolyte solution in order to enhance the sensitivity of the electrochemical assay. In some embodiments, the electrochemically active moiety is a sacrificial electron donor. Suitable sacrificial electron donors include, but are not limited to, disodium ethylenediaminetetraacetic acid (EDTA), triethanolamine (TEOA), triethylamine (TEA), and tripropylamine (TPA). In some embodiments, the sacrificial electron donor is EDTA, which is dissolved in the redox electrolyte solution. In some embodiments, the electrolyte solution in which the EDTA is dissolved further comprises a suitable solvent, examples of which include but are not limited to water, alcohol, acetonitrile and CH₂Cl₂.

Detection can be achieved by irradiating the electrodes individually with a light source. Light sources can comprise, for example, a tungsten halogen light source, a xenon arc lamp or a laser (e.g., a YAG laser).

In yet another embodiment, the exposing is by rastering, and the exposing and the detecting are performed simultaneously, as set forth in more detail below.

In some embodiments, a light source can be configured so as to allow irradiation of samples individually and sequentially, for example when a plurality of samples is being scanned. When a laser is used, the beam can be rastered across the support in a predictable pattern, such as horizontally or vertically. The rastering motion can be staggered so as to permit irradiation and detection of a current carried by a given sample (e.g., at an attachment point at which a nanoparticle-comprising hybridization complex was formed), before a subsequent (e.g., sequential) sample is irradiated and monitored for the presence of a current. In one example, a light source, such as a rastering laser beam, can be used to irradiate discrete points on the support, correlating to the attachment points of the capture probes on the support. Irradiation generates a current that is detected by monitoring the current through the support electrode. Each array attachment point (and therefore each potential site of hybridization complex formation) is irradiated, and any generated current detected, in a sequential fashion, as can be accomplished through the use of a rastering light source.

For example, photoinduced electron transfer can be measured by focusing a laser on one particular spot of the microarray (corresponding to a particular labeled probe that is attached at that point) and poising the potential of the electrode at a value where no current should flow in the dark, but where current will flow in the presence of light. In this way, the methods described herein eliminate the need to individually wire each spot or area of the array to detect a hybridization event electrochemically, by employing a single electrode.

The artisan can select the wavelength at which to irradiate the nanoparticles attached to the electrode based on the material comprising the nanoparticle or nanoshell. As provided above, in some embodiments the wavelength of light matches the surface plasmon resonance of the material that comprises the nanoparticle. In another embodiment, the wavelength of the irradiation matches another wavelength that is absorbed by the nanoparticle (e.g., a wavelength at which a nanoparticle metal component undergoes interband transition). By “matching” is meant that the wavelength of light is identical to (i.e., equivalent) or is nearly equivalent to a light wavelength known to be absorbed by the nanoparticle, which absorbance causes the nanoparticle to generate heat. The particular wavelength will be determined by the material comprising the nanoparticle, and can also be dependent on the shape of the nanoparticle, the size (i.e., diameter) of the nanoparticle, and the thickness of the material of the nanoparticle (e.g., in particular, if the nanoparticle is a nanoshell). However, the ability to calculate the surface plasmon resonance based on these factors is within the skill of the artisan. For example, if the nanoparticle comprises gold, the wavelength of light used to irradiate the nanoparticle will generally be in the range of about 510 nm to about 560 nm, more usually in the 520-530 nm range, and in some embodiments about 532 nm. If the nanoparticle is silver, the light wavelength will generally be in the 420-460 nm range. In contrast, if the nanoparticle comprises a metal oxide, the wavelength of the exciting light will generally be in the near-infrared range The detection of the electronic signal associated with the oxidation-reduction reaction permits the determination of the presence or absence of hybridized DNA. For example, determining the presence or absence of hybridized DNA can include (i) measuring the generation of current by the photoinduced oxidation-reduction reaction and then (ii) comparing the generated current to the current generated by the complex-free electrode. Alternatively or in addition, determining the presence or absence of hybridized DNA can include (i) measuring the potential of the electrode supporting the photoinduced oxidation-reduction reaction and then (ii) comparing the potential to the potential of the complex-free electrode (i.e., an electrode without attached nanoparticles), for example.

The detected signal can also be compared to a predetermined threshold or control. The control can be any appropriate control, such as a control under substantially the same conditions, except that no nucleic acids are present, or only non-target sequences are present.

In some embodiments, a competitive assay format is provided. Unlabeled sample target sequences compete with a predetermined amount of competitive, labeled sequences, for hybridizing to capture probes.

Measuring the current or potential can be carried out by any suitable means. In some embodiments, the oxidation-reduction reaction is measured by measuring the electronic signal associated with the occurrence of the oxidation-reduction reaction. For example, the electronic signal associated with the oxidation-reduction reaction can be measured by providing a suitable apparatus in electronic communication with the detection electrode. A suitable apparatus will be capable of measuring the electronic signal that is generated so as to provide a measurement of the oxidation-reduction reaction of the hybridization complex and the redox solution. A positive current flow is indicative of attachment (e.g., hybridization complex formation). The current is detected and compared with an amount of current that is generated by a complex-free electrode.

Detection of generated electric current is carried out using one of any number of suitable means, including amperommetry, voltammetry, and capacitance and impedence detection techniques. Suitable techniques include, but are not limited to, electrogravimetry; coulometry (including controlled potential coulometry and constant current coulometry); voltammetry (cyclic voltammetry, pulse voltammetry (normal pulse voltammetry, square wave voltammetry, differential pulse voltammetry, Osteryoung square wave voltammetry, and coulostatic pulse techniques); stripping analysis (anodic stripping analysis, cathodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; and capacitance measurement.

A general definition for the term “voltammetry” is any electrochemical technique that involves controlling the potential of an electrode while simultaneously measuring the current flowing at that electrode. In voltammetry, current at a working electrode in solution is measured as a function of a potential waveform applied to the electrode. The resulting current-potential curve is called a voltammogram, and correct interpretation provides information about the reaction occurring at the surface of the electrode.

Voltammetry is usually performed by connecting an electrochemical potentiostat to an electrochemical cell. The cell contains a test solution and three electrodes. One of the three electrodes is the working electrode. The second electrode is a reference electrode, against which the potential of the working electrode is measured. The third electrode is called a counter electrode. The counter electrode is usually a piece of inert, conducting material such as Ag or Pt.

A device, generally a potentiostat, controls the potential of the working electrode. It is designed to work with a three electrode cell in a way which assures that all current will flow between counter and working electrodes, while controlling the potential of the working electrode with respect to the reference electrode. Special electronic circuitry within the potentiostat permits the working electrode potential to be controlled with respect to the reference electrode without any appreciable current flowing at the reference electrode. The simplest potentiostat has a means of setting the starting potential and the switching potential, a sweep rate adjustment, and outputs which monitor working electrode potential and current flow. These are connected to the X and Y axes of an X-Y recorder, respectively. Modern electrochemical systems are often “closed-box” systems that are controlled by a computer.

In a typical voltammetric experiment, oxidation or reduction of analytes occurs at the surface of a working electrode when the electrode is biased near the redox (Nernst potential) of a given analyte. At this potential, electron transfer takes place and a measurable change in current occurs whose magnitude is linearly proportional to the concentration of the given analyte in solution. Therefore, the magnitude of the current peak provides concentration information while the potential at which the current peak occurs identifies the analyte. Additional information such as the reaction type (reversible, quasi-reversible, and irreversible) and analyte mass-transport rates (diffusion coefficients) can also be obtained depending on the type of voltammetric experiment performed. Since most analytes have different redox potentials, voltammetry allows the measurement of multiple analytes in solution.

There are several variations of voltammetric measurements, and most of these are due to changes in the type of potential waveform (i.e., input/probe signal and/or shape of input/probe signal used to sweep the voltage range) used (e.g., cyclic, staircase, AC, squarewave, pulse, and differential pulse voltammetry) and/or the addition of a preconcentration step (stripping voltammetry). Consequently, the choice of technique determines how many characteristics of the redox reaction can be measured and how well a given characteristic can be measured.

The shape of a voltammogram gives information about the kinetics of electrode processes. The shape of the current peaks due to the “Faradaic processes” (this terminology is used to denote charge transfer processes) is determined by the concentration of the redox species at the electrode surface. In cyclic voltammetry the electrolyte solution is not stirred, and it is important that the system is at rest (i.e., no mechanical agitation) while the experimentation is performed. Under these conditions the surface concentration is governed by diffusion of the redox active species to the electrode surface.

For example, in cyclic voltammetry, a DC voltage sweep is done. In AC voltammetry, an AC signal is superimposed on to the voltage sweep. In square wave voltammetry, a square wave is superimposed on to the voltage sweep. Most preferably, the signal is recorded from each position (“address”) on an array (e.g., at one attachment point on an array).

In cyclic voltammetry, the voltage that is applied to the working electrode is an inverted triangle wave, so that the electrode potential becomes more negative linearly in time until it reaches a predetermined switching potential, at that point the potential of the working electrode is scanned to more positive potentials, again varying linearly in time. In cyclic voltammetry, the working electrode potential is swept back and forth across the formal potential of the analyte. Cyclic voltammograms trace the transfer of electrons during a redox reaction. The reaction begins at a certain potential (voltage). As the potential changes, it controls the point at which the redox reaction will take place. Repeated reduction and oxidation of the analyte causes alternating cathodic and anodic currents flow at the electrode.

Experimental results are usually plotted as a graph of current versus potential. The voltammogram exhibits two asymmetric peaks, one cathodic and the other anodic. The signal of primary interest to the artisan will be the height of the peak or peaks. The voltammogram can provide information about both the oxidation and reduction reaction which includes the thermodynamics of the redox processes, the kinetics of heterogeneous electron transfer reactions, analyte identification and quantitation, and analyte diffusion coefficients.

Cyclic voltammetry (CV) is advantageously used to study the electroactivity of compounds, particularly biological molecules. In particular, it is well suited to probe-coupled chemical reactions, particularly to determine mechanisms and rates of oxidation/reduction reactions. Moreover, cyclic voltammetry can be used to study electrode surfaces and the reactions that take place thereon.

Stripping voltammetry techniques such as anodic stripping voltammetry, cathodic stripping voltammetry, potentiometric stripping analysis and adsorptive stripping voltammetry can also be used with the present methods.

In addition to voltammetry, other methods such as chronoamperometry can be used. In chronoamperometry, the working electrode potential is suddenly stepped from an initial potential to a final potential, and the step usually crosses the formal potential of the analyte. The solution is not stirred. The initial potential is chosen so that no current flows (i.e., the electrode is held at a potential that neither oxidizes or reduces the predominant form of the analyte). Then, the potential is stepped to a potential that either oxidizes or reduces the analyte, and a current begins to flow at the electrode. This current is quite large at first, but it rapidly decays as the analyte near the electrode is consumed, and a transient signal is observed.

In an integrated configuration, light sources and signal processing (detection devices, voltage source and current meters or other voltage sources and current meters) can be integrated all on the same configuration, device or chip. Alternatively or in addition, the signal processing can be done off separately.

In some embodiments, cyclic voltammetry is used to measure the current in the electrode, and the apparatus used comprises a plurality of electrodes, including the electrode upon which the hybridization reactions are carried out (i.e., the working electrode), at least one counter-electrode and optionally a reference electrode, and an electrolyte solution in contact with the plurality of microelectrodes, counter electrode and reference electrode. The working electrode can, as set forth above, be supported by another solid substrate. The solid substrate can comprise one working electrode, or a plurality of working electrodes. In some embodiments, a solid substrate can comprise a plurality or microelectrodes on its surface.

In some embodiments, the hybridization reactions set forth above are carried out in a reaction chamber located within a suitable electrochemical cell. Following hybridization of a target probe to an array of capture probes on an electrode surface, and hybridization of detection probes to any captured target sequences on the electrode, the electrodes are thoroughly rinsed in an excess volume of buffer, generally at room temperature. After washing, a suitable volume of a redox solution, as set forth above, is added to the reaction chamber, and each working electrode is interrogated by conventional cyclic voltammetry to detect a redox signal. The reaction chamber can optionally comprise at least two compartments, the working electrode compartment and the counter electrode compartment. The counter electrode compartment can be separated from the working electrode compartment by means of a gas permeable separator, which allows passage of a buffer solution and gases between the compartments, but does not permit passage of the reactants, e.g., the redox mediator. Suitable gas permeable separators can be made, for example, from glass, dialysis membranes, and Teflon-based materials, such as Nafion.™

The counter electrode can be made of any suitable material that is noncorrosive in the electrochemical cell and reaction solutions utilized. A preferred counter electrode is made of a material that is capable of supplying oxygen or hydrogen to the reaction vessel during the reaction, such as a platinum group metal, a metal oxide, and/or a carbon-based material. Representative counter electrode materials include palladium; ruthenium; platinum as wires, sheets or thin films; ruthenium oxide; glassy carbon; reticulated carbon; titanium dioxide; and mixed metal oxides.

Any suitable reference electrode can be used, such as a Ag/AgCl electrode, a calomel reference electrode or a normal hydrogen electrode.

J. Uses and Advantages of Methods

In a broad aspect, the methods described herein concern an electrochemical system for detecting specific target sequences by the use of oligonucleotide probes that are specific for identifying segments of such acids. These methods have applications in regard to detecting identified nucleic acids in complex mixtures, and are particularly useful for assaying virtually any species so long as an identifiable sequence can be determined. Diagnostic assays, such as for aberrant chromosomal variations, cancers and genetic abnormalities are facilitated by methods described herein to the extent that targeted nucleic acid sequences or segments can be selectively probed employing the described methods.

The described methods can be employed to detect hybridization on an array and can be employed, for example, in sequencing, in mutational analysis (single nucleotide polymorphisms and other variations in a population), and for monitoring gene expression by analysis of the level of expression of messenger RNA extracted from a cell. Thus, examples of the uses of the methods of detecting nucleic acids include the diagnosis and/or monitoring of viral and bacterial diseases, inherited disorders, and cancers where genes are associated with the development of cancer; in forensics; in DNA sequencing; for paternity testing; for cell line authentication; for monitoring gene therapy; and for many other purposes.

Moreover, methods described herein can be employed to monitor hybridization events in a variety of different systems and models. As described more fully below, the present methods can be used in the monitoring of gene expression, the detection of spontaneous or engineered mutations and in the design of probes.

In some embodiments, the present methods can be used to monitor gene expression. In some embodiments, single stranded DNA derived from a gene of interest is used as capture probe. Unexpressed sequences of DNA (for example introns) can be removed before the samples are attached to the support. In this application, it can be desirable to employ cDNA as a probe sequence. Control samples of unrelated single-stranded DNA can also be included to serve as an internal validation of the experiment.

Total mRNA is then isolated from an expression system using standard techniques, which mRNA serves as the target nucleic acid. Target mRNA can optionally be fragmented for ease of handling. The target mRNA is hybridized to the capture probe as described herein. A detection probe comprising a nanoparticle-oligonucleotide complex is then contacted with the support-bound target. In some embodiments of the method, conditions of high stringency are maintained, although these conditions can be varied with the needs and goals of the experiment. The electrode can be washed to remove any unhybridized sample.

The electrode is then irradiated by a light source, such as a laser. Electrons transferred from the nanoparticle to the electrode are detected by monitoring current flow in the electrode. Gene expression can be determined by comparing duplex formation by the control sequences to duplex formation observed in the target samples. Appropriate mathematical descriptions and treatments of the observed duplex formation can indicate the degree of observed hybridization and consequently the degree of gene expression.

In some embodiments, the present methods can also be employed in the detection of mutations in a nucleic acid sequence. Such mutations can engineered or spontaneous. For example, the present methods can be useful in determining whether an engineered mutation is present in a nucleic acid sequence, or for determining if a nucleic acid sequence contains deviations from its wild type sequence.

In these embodiments, single-stranded oligonucleotide probes are initially prepared. The probes can be known or suspected to contain a mutation(s) to be identified. Capture probe samples are attached to the support using methods described herein. Nucleic acid target sequences to be screened for the mutation are isolated from an expression system, and single stranded target sequences are prepared. If desired, large quantities of sample can be conveniently prepared using established amplification methods, as set forth above. Probe sequences are bound to a nanoparticle to form a detection probe, which is contacted with the capture probe-target hybridization complexes. Those probe sequences containing the mutation of interest will hybridize with the target sequence to form detectable complexes. Unbound target sequences can be removed by washing. The support, which can comprise any formed duplexes, is then irradiated by light and the resulting photocurrent detected. In this embodiment, a mutation can be located on either a target sequence or on a probe sequence, the selection of which can be made during experimental design.

In some embodiments, the present methods can be employed in designing nucleic acid probes. The ability to detect hybridization events permits a researcher to optimize a probe for the needs of a given experiment. For example, a probe can be designed that will accommodate a degree of polymorphism in a target sample. Such a probe can be useful for screening for genes or sequences known to exhibit polymorphisms. Using the presently disclosed subject matter, it is possible to design a probe that will tolerate a degree of uncomplementarity in the sequence.

Additionally, the present methods can be used to screen for duplex formation between a target sequence and a polymorphic probe; that is, a probe that has one or more mutations from the wild type sequence. By varying the number of bases different from the wild type sequence, a desired degree of promiscuity in a probe can be obtained.

In this context, the present methods can be useful for detecting hybrid formation in sequential rounds of probe design. For example, if a designed probe binds only to the wild type sequence, no polymorphism is recognized; if the probe binds to sequences unrelated to the target sequence, the probe is not useful to identify the sequence of interest. By monitoring hybrid formation at each round of optimization, the presently disclosed subject matter can be useful for nucleic acid probe design.

Photocurrent detection methods of the presently disclosed subject matter offer significant advantages over detection systems known in the art. One particular advantage is the elimination of any requirement for individually wired sample cells. Commercially available microarray supports suitable for electrochemical detection of nucleic acid duplexes require that each sample be attached to the support at a different electrode. That is, duplex formation at each attachment point must be monitored by detecting a current through an electrode dedicated to each individual cell. The presently disclosed subject matter can employ in some embodiments only a single electrode and achieves detection at each capture probe attachment point on the electrode by detecting current flow following irradiation of each capture probe attachment point by a light source, for example a laser beam.

### EXAMPLES

The following Examples have been included to illustrate some modes of the disclosed subject matter. Certain aspects of the following Examples are described in terms of techniques and procedures found or contemplated by the present inventors to work well in the practice of the disclosed subject matter. In light of the present disclosure and the general level of skill in the art, those of skill will appreciate that the following Examples are intended to be exemplary only and that numerous changes, modifications and alterations can be employed without departing from the spirit and scope of the disclosed subject matter.

Examples 1-5 illustrate the effects of laser-induced temperature jumps (LITJ) on the potential of gold nanoparticle-coated indium tin oxide (ITO) electrodes in contact with electrolyte solutions.

### Example 1

### X-Ray Photoelectron Spectroscopy Characterization of ITO Electrode Surfaces Modified by Single Stranded DNA and Gold Nanoparticles

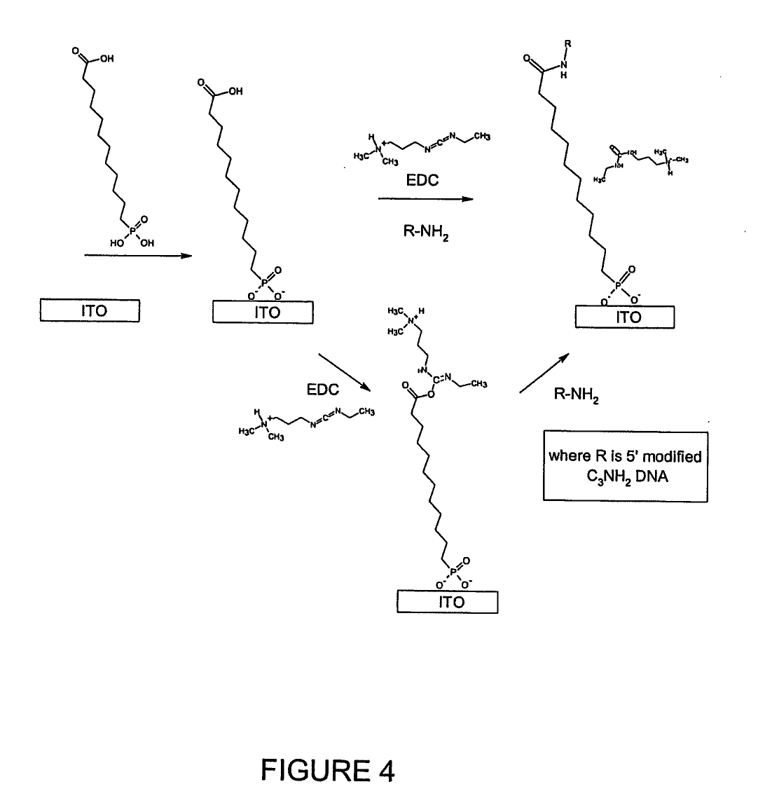

FIG. 4 outlines one strategy employed in the modification of indium tin oxide (ITO) with single-stranded DNA (ssDNA). Initially, a monolayer of 12-phosphonododecanoic acid (10 mM in 50/50 DMSO/18 MΩ cm H₂O for 16 hours) was formed on the ITO surface (cleaned 20 minutes with UV/O₃ (UVO-cleaner (UVO-60), model number 42, Jelight Company, Inc.)). The carboxylic acid of 12-phosphonododecanoic acid was then activated by 1-ethyl-3-(3-dimethylaminopropyl)carbodiimide hydrochloride (EDC) to form an O-acylisourea intermediate. See, e.g., S. H. Brewer et al., Langmuir (2002) 18, 6857-6865; B. L. Frey and R. M. Corn, Analytical Chemistry (1996) 68, 3187-3193; M. Burgener et al., Bioconjugate Chemistry (2000) 11, 749-754; K. Kerman et al., Analytica Chimica Acta (2002) 462, 39-47; E. Huang et al., Langmuir (2000) 16, 3272-3280; and G. T. Hermanson, Bioconjugate Techniques (1996) (Academic Press: San Diego).

This activated carboxylic acid group is attacked by the primary amine (acting as a nucleophile) of a 5′-modified C₃NH₂ ssDNA strand to form an amide bond between the monolayer of 12-phosphonododecanoic acid and the 5′ modified C₃NH₂ ssDNA. The coupling conditions were 1 μM 5′-modified C₃NH₂ ssDNA and 200 mM EDC for 4 hours in a 0.1 M MES (2-(N-morpholino)ethane sulfonic acid) buffer at pH 5 with 0.25M NaCl.

X-ray photoelectron spectroscopy (XPS) spectra were recorded on a Riber LAS 2000 Surface Analysis System equipped with a cylindrical mirror analyzer (CMA) and a MAC2 analyzer with Mg Kα X-rays (model CX 700 (Riber source) (hν=1253.6 eV). The elemental scans had a resolution of 1.0 eV and were the result of 5 scans. XPS spectra were smoothed using a 9 point (second order) Savitzky-Golay algorithm, baseline corrected and the peaks were fitted using Gaussian line shapes.

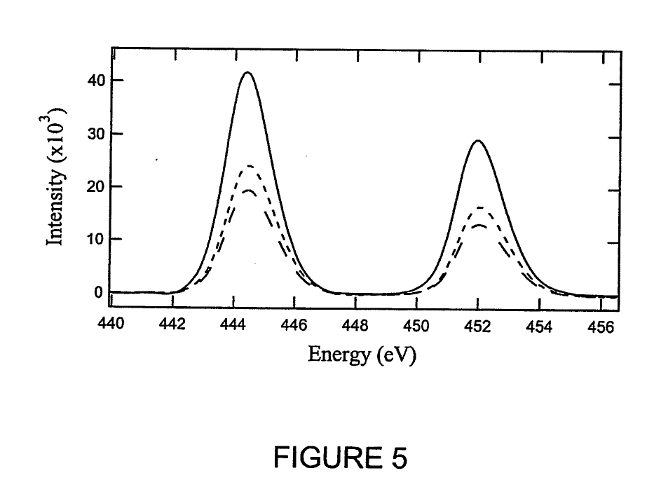

The results of these experiments are shown in FIGS. 5, 6, 7 and 8.

FIG. 5 is the x-ray photoelectron spectra (XPS) of In 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

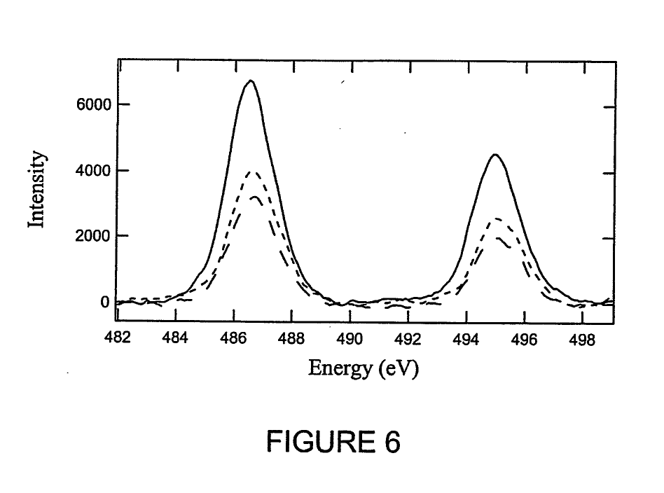

FIG. 6 is the XPS spectra of Sn 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

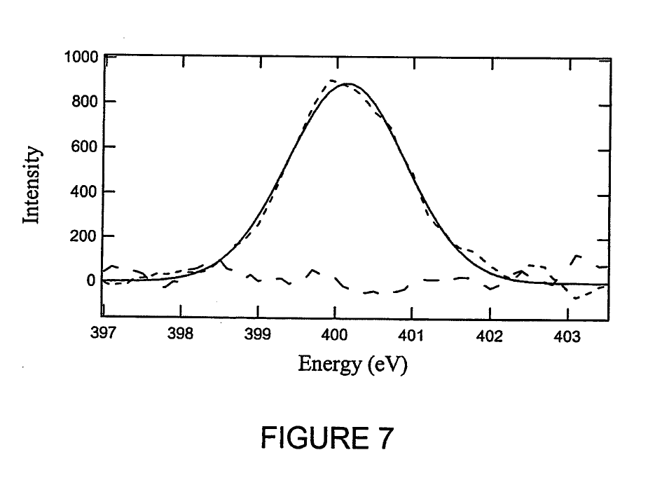

FIG. 7 is the XPS N 1 s spectra of ITO modified with a monolayer of 12-phosphonododecanoic acid (long dash) and ITO modified with ssDNA coupled through a monolayer of 12-phosphonododecanoic acid (short dash) fitted to a Gaussian line shape (solid).

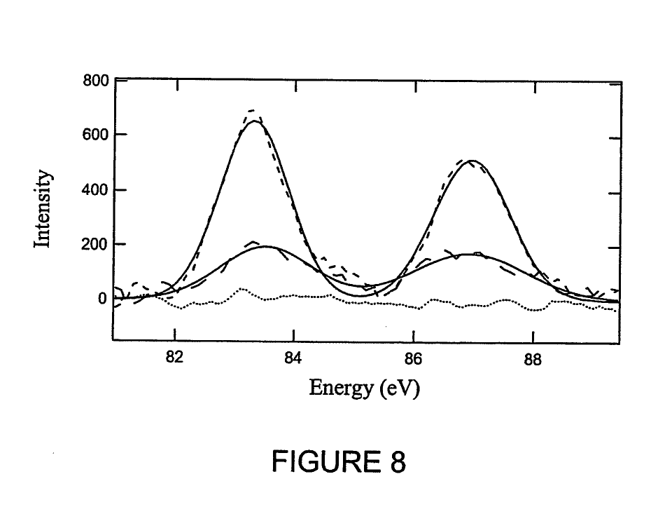

FIG. 8 is the XPS Au 4f_{7/2,5/2} spectra of ITO modified with ssDNA coupled through a monolayer of 12-phosphonododecanoic acid (dotted line) exposed to the complementary (short dash) or non-complementary (long dash) ssDNA labeled with a 10 nm gold nanoparticle (1 nM) fitted to two Gaussian line shapes (solid).

### Example 2

### Infrared Reflection Absorption Spectroscopy (IRRAS)

The reflectance FTIR spectra were recorded using a Spectra-Tech grazing angle reflectance attachment in a Nicolet Magna-IR 860 FTIR spectrometer. The angle of incidence used was 80 degrees. An infrared polarizer was used to p- (vertically) polarized light. The spectra of the monolayers deposited on the ITO surfaces were obtained by taking a ratio of the single beam spectra of the deposited material on an ITO surface to one of a clean ITO surface. The rotational lines from gaseous water were subtracted from these spectra. The FTIR spectrometer was equipped with a liquid nitrogen cooled MCT/A detector and the spectra were recorded at a resolution of 2 cm⁻¹ with a spectral range of 900-4000 cm⁻¹. All IR spectra were the result of 256 scans and were recorded at room temperature.

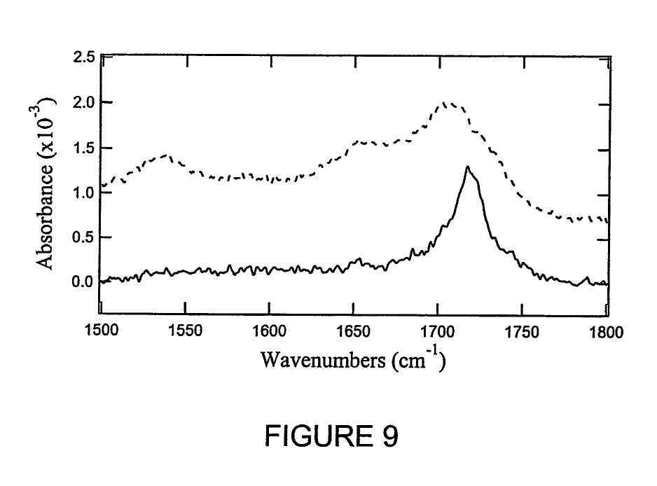

The results of these experiments are illustrated in FIG. 9. FIG. 9 shows a grazing angle reflectance FTIR spectra of ITO modified with a monolayer of 12-phosphonododecanoic acid (solid) coupled to ssDNA (dashed) recorded at an incident angle of 80 degrees with p-polarized radiation.

### Example 3

### LITJ Electrochemistry at Gold Nanoparticle-Coated ITO Electrodes

LITJ was demonstrated by attaching 10 nm diameter gold particles to ITO via aminosilane linkers, according to the method of K. C. Grabar et al., J. Am. Chem. Soc. (1996) 118, 1148. Visible spectroscopy revealed a particle coverage of 1.5×10¹⁰/cm². Current was then monitored during illumination with 532 nm light from a frequency doubled YAG laser (Coherent Antares 76-YAG laser).

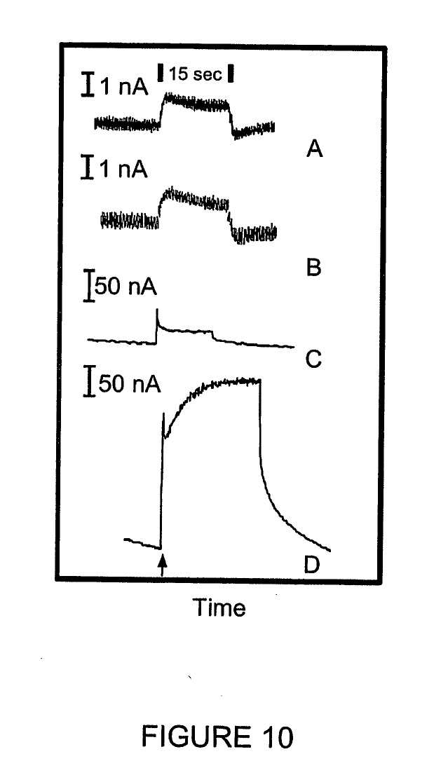

FIG. 10 is a graphical illustration of anodic current vs. time for ITO electrodes in (A) 0.1 M phosphate buffer (pH 7.3), (B) 0.1 M phosphate buffer/0.05 M EDTA, and (C) 0.1 M phosphate buffer following adsorption of 10 nm diameter gold particles to the electrode via aminosilane linkers. FIG. 10(D) illustrates the conditions of the electrode from FIG. 10(C) with 0.05 M EDTA added to solution. The arrow indicates the start of an approximately 15 second laser irradiation cycle with 532 nm light (0.64 W/cm²). The potential was held at 0.3 V vs. Ag₍ₛ₎/AgCl.

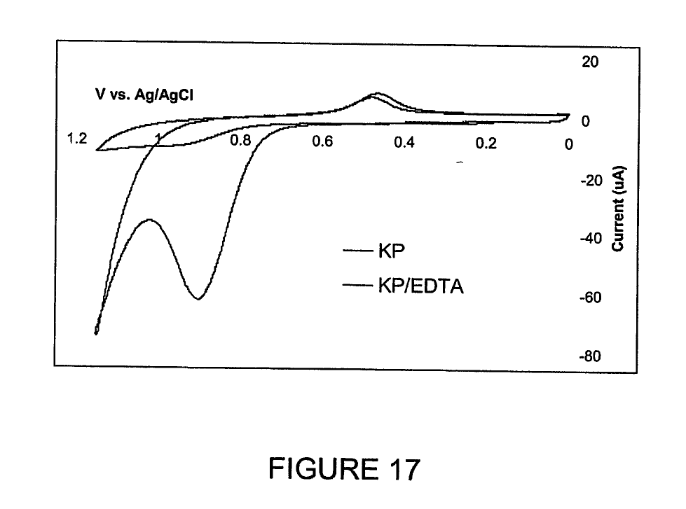

Overall, FIG. 10 shows that the largest anodic current was passed when gold nanoparticles were bound to the electrode surface and the electroactive molecule EDTA was present in solution. Further confirmation of the detection enhancement effect of adding EDTA to the electrolyte solution is shown in FIG. 17. FIG. 17 is a graph comparing the cyclic voltammogram trace of gold nanoparticles hybridized onto ITO electrodes when the electrode solution comprises an electrolyte solution without EDTA (KP, upper trace/small current peak observed) and with EDTA (KP/EDTA, lower trace/large current peak observed).

### Example 4

### Detection of Temperature Change at Electrode

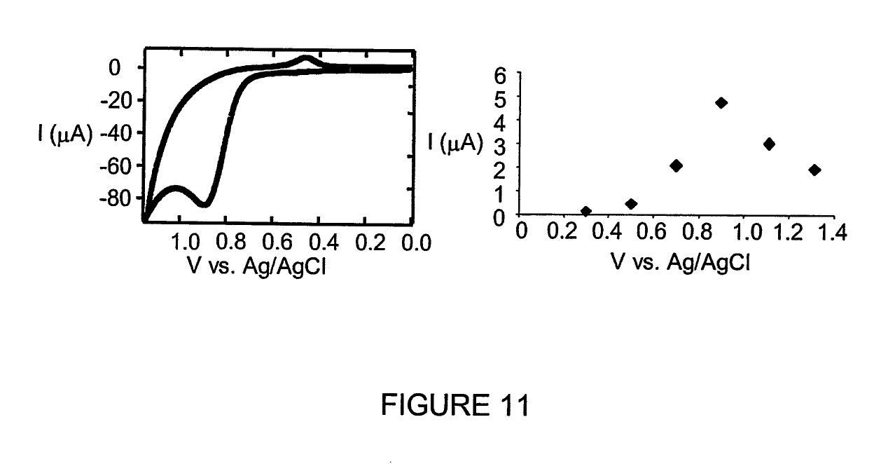

Close inspection of the data shown in FIG. 10D revealed an initial anodic current spike followed by a second rise in anodic current. The thermodynamic parameter ΔV_{redox} was assigned to the initial spike, and the second anodic current increase to faster electron transfer kinetics at the higher temperature. The photocurrent was observed to increase with applied potential, approaching a maximum value near the oxidation peak potential of EDTA at gold nanoparticles (0.9 V vs. Ag₍ₛ₎/AgCl), as shown in FIG. 11, which is a cyclic voltammogram (left) and a graph of photocurrent vs. applied potential (right) for EDTA on gold-nanoparticle-coated ITO electrodes.

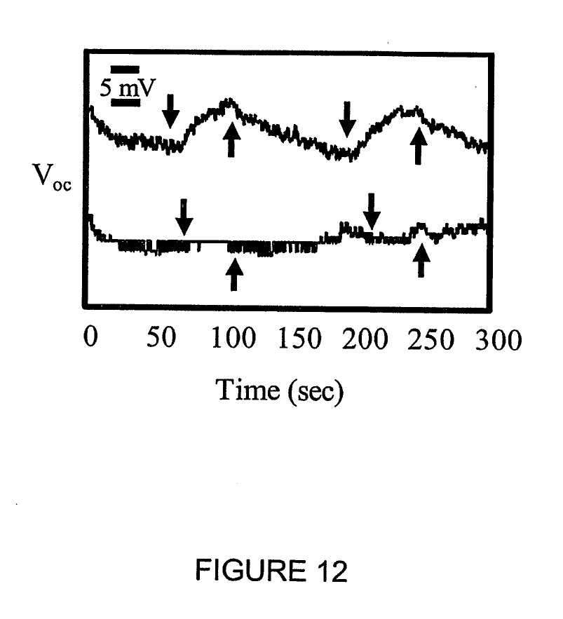

The temperature change at the electrode surface was measured using an internal standard consisting of 100 mM ferrocene and 0.1 mM ferrocinium in acetonitrile (Aldrich). The temperature dependence of this redox couple was first measured with a 2-compartment electrochemical cell and hot plate to be 0.35 mV ° C.⁻¹. When the solution was placed in contact with a gold nanoparticle-coated ITO electrode and irradiated at 532 nm, a 9 mV change was recorded, as illustrated in FIG. 12. This value corresponds to an interfacial ΔT of 25° C. induced by the LITJ effect.

FIG. 12 is a graphical illustration of open circuit voltage vs. time for ITO electrodes in contact with 100 mM ferrocene and 0.1 mM ferrocinium in acetonitrile/0.1 M NaClO₄. The electrode in the top curve contained 1.5×10¹⁰ gold nanoparticles cm⁻². The bottom curve was ssDNA-coated ITO. Downward and upward arrows indicate light on and off, respectively. In FIG. 12, the curves were offset for clarity.

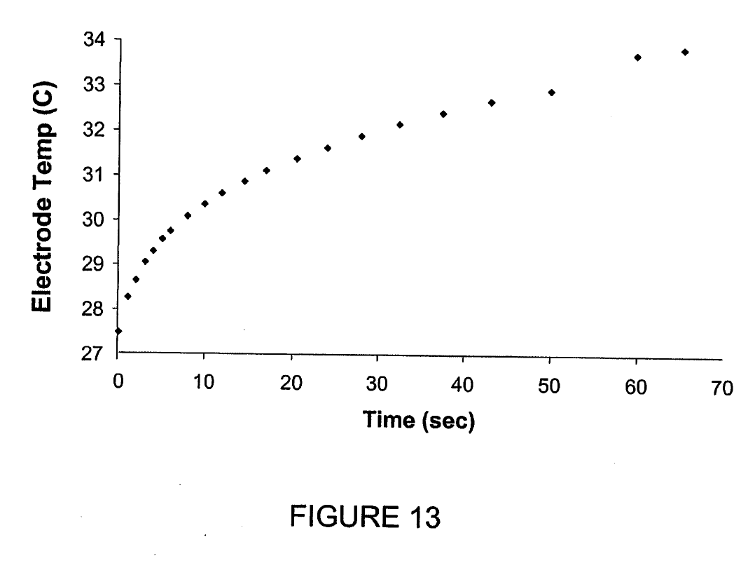

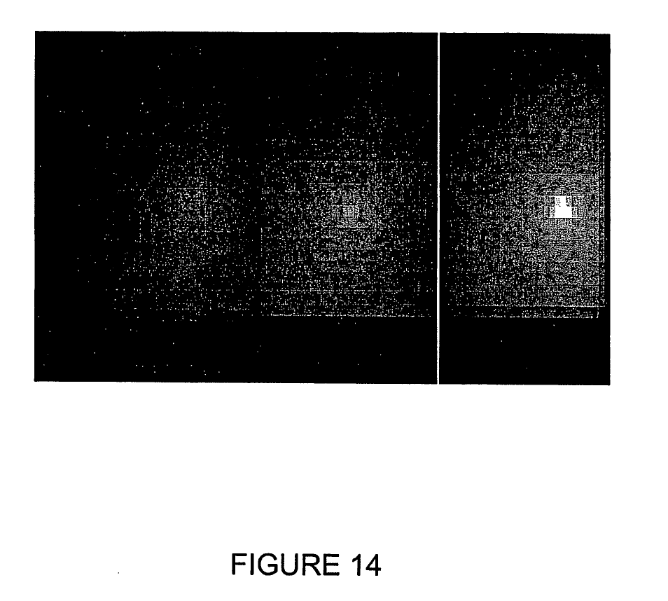

The temperature change at the electrode surface was confirmed by infrared thermography (Inframetrics Inc., Model 740). Following 30 seconds of irradiation, a surface temperature of 42.9° C. was measured for a particle coverage of 3.5×10¹⁰ particles cm⁻². FIG. 13 is a graph showing the increase in electrode temperature as a function of time. FIG. 14 shows a series of infrared thermograms (8 μm-12 μm) of gold nanoparticle-coated glass slides under irradiation with 532 nm light (16 W/cm²). Particle densities were 1×10¹⁰ cm⁻², 2×10¹⁰ cm⁻², and 3.5×10¹⁰ cm⁻² for A, B, and C, with recorded temperatures of 30.5° C., 35.3° C., and 42.9° C., respectively. Light-off temperature was 24.6° C. (ΔT for bare glass was <2° C.). In other experiments, temperature changes of 2.5° C. for as few as 10,000 nanoparticles (10⁶ cm⁻²) have been recorded using IR thermography.

### Example 5

### Photoelectrochemical Detection of Nucleic Acid Hybridization at Gold-Nanoparticle-Coated ITO Electrodes

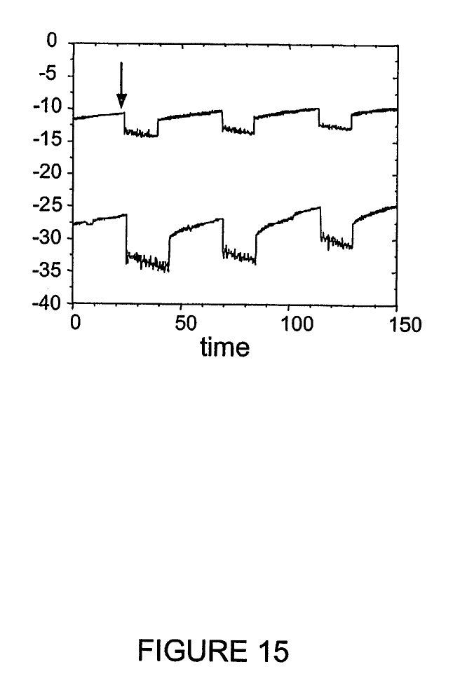

Complementary 18-base pair single-stranded DNA sequences were attached to ITO and 10 nm diameter gold nanoparticles according to the methods set forth in Example 1. FIG. 15 illustrates shows that hybridization events between nanoparticles and the surface can be detected with the LITJ photoelectrochemical response. FIG. 15 is an illustration of anodic current vs. time for an ITO electrode in 0.1 M phosphate buffer/0.05 M EDTA following adsorption of 10 nm diameter gold particles to the electrode via DNA hybridization. The potential was held at 0.5 V vs. Ag/AgCl. The bottom current trace represents ssDNA/gold nanoparticle conjugates hybridized from a 100 fM solution as described in M. L. Sauthier. et al., (2002) Langmuir 18, 1825 and S. H. Brewer, et al., (2002) Langmuir, 18, 6857-6865. The top trace represents ssDNA probe strands on ITO. The arrow indicates light on. The current signal in the bottom trace is ˜2×background current of top trace.

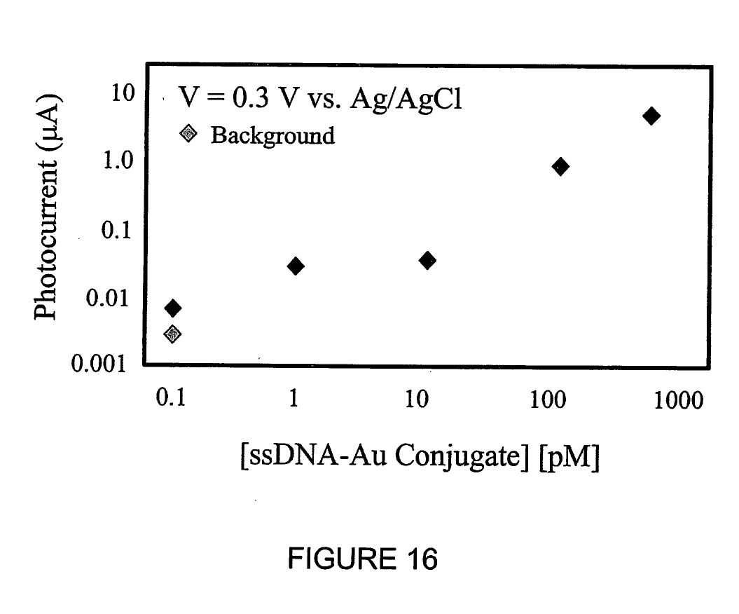

Surface hybridization has been detected with signals at least twice background for ssDNA-modified gold nanoparticle solution concentrations ranging from 100 fM to 1.0 nM, as illustrated in FIG. 16. FIG. 16 is an illustration of the limits of detection of methods of the presently disclosed subject matter. The striped points indicate background current, while solid points represent the detected current as a function of concentration in (pM) of ss-DNA-conjugated gold nanoparticles. The present methods can are able to detect (distinguish over background) hybridization of nucleic acids at electrode surfaces in concentrations as low as 0.1 pM.

The foregoing examples illustrate that laser-induced temperature jumps (LITJ) at gold particle-coated indium tin oxide (ITO) electrodes in contact with electrolyte solutions have been measured using temperature-sensitive redox probes. Upon irradiation with 532 nm light, interfacial temperature changes of ca. 20° C. were recorded for particle coverages of ca. 1×10¹⁰ cm⁻². In the presence of a redox molecule, LITJ yields open-circuit photovoltages and photocurrents that are proportional to the number of particles on the surface. When ssDNA was used to chemisorb nanoparticles to the ITO surface, solution concentrations as low as 100 fM of target ssDNA-modified nanoparticies can be detected at the electrode.

### Example 6

### Exemplary Thermographic Data

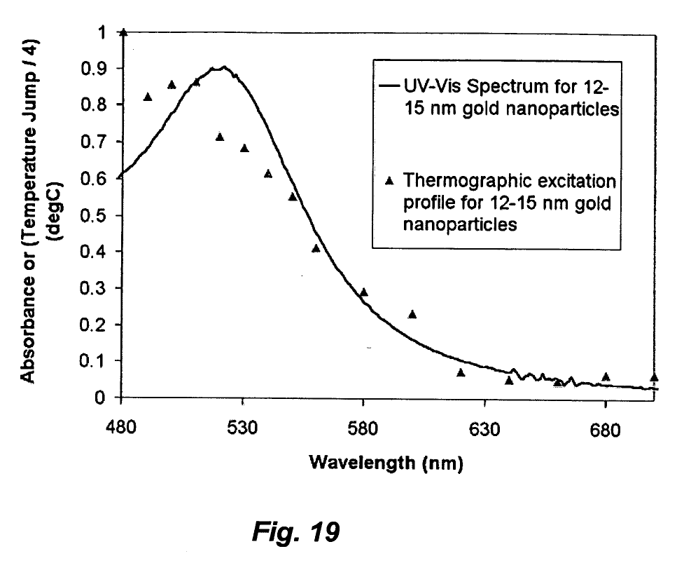

A representative thermographic excitation profile for 12- to 15-nm gold nanoparticles is provided in FIG. 19. The data provided in FIG. 19 demonstrate that the heat released, for example, by a gold nanoparticle, upon excitation is directly related to the absorbance spectrum of the gold nanoparticle. The solid line of FIG. 19 represents the UV-Vis spectrum for 12- to 15-nm gold nanoparticles, whereas (A) represents the thermographic excitation profile for 12- to 15-nm gold nanoparticles.

Further, the temperature change measured in the thermographic detection of nucleic acids can be a linear function of the nanoparticle density. More particularly, the data provided in Table 1 show the temperature change as a function of nanoparticles on the surface of an electrode.

TABLE 1
Temperature Change as a Function of Nanoparticle Density

| | | | | Temperature |
|---|---|---|---|---|
| Amount of | Density of | | Temperature | Increase |
| Nanoparticles | Particles | Background | after 30 | After 30 |
| per Spot | (particles per | Temperature | Seconds | Seconds |
| (amoles) | μm²) | (° C.) | (° C.) | (° C.) |
| 0 | 0 | 22.6 | 22.8 | 0.2 |
| 0.33 | 0.028 | 22.6 | 23.0 | 0.4 |
| 3.3 | 0.28 | 22.4 | 23.9 | 1.5 |
| 33 | 2.8 | 22.1 | 27.4 | 5.3 |
| 330 | 28 | 21.5 | 57.4 | 35.9 |

Conditions: 30-nm citrate-coated gold nanoparticles; Spot size: 3 mm in diameter; Temperature read after 30 second illumination; Coherent Antares laser at 532 nm; Laser power: 14.1 W/cm²

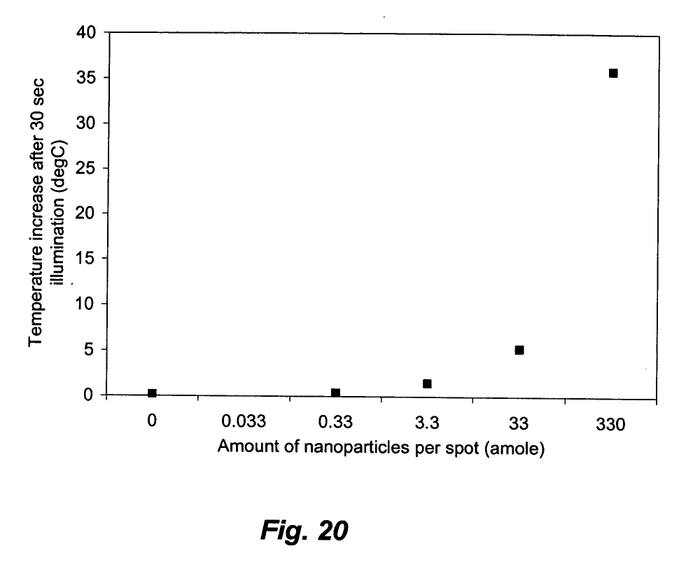

The linear relationship between the temperature change and the nanoparticle density is further illustrated in FIG. 20, which plots the temperature increase after 30 seconds of illumination (in ° C.) vs. the amount of nanoparticles per spot (in amole) (plotted in log scale). The linear fit (R²) for the data provided in FIG. 20 is 99.8%.

The influence of laser power on the temperature increase is demonstrated by the data provided in Table 2. The data in Table 2 demonstrate that the magnitude of the temperature change can increase as a function of an increase in laser power.

TABLE 2
Influence of Laser Power on the Temperature Increase

| | Temperature (° C.) | Temperature (° C.) |
|---|---|---|
| Amount of | Increase after 30 | Increase after 30 |
| Nanoparticles per | Seconds at 127 W/cm ² | Seconds at 178 W/cm² |
| Spot (amole) | Laser Power | Laser Power |
| 0 | 0.3 | 0.3 |
| 0.0033 | 0.3 | 0.5 |
| 0.033 | 0.4 | 1.1 |

Conditions: 30 nm citrate-coated gold nanoparticles; Spot size: 1 mm in diameter; Temperature read after 30 second illumination; Coherent Antares laser at 532 nm; Laser power: 127 W/cm² or 178 W/cm²

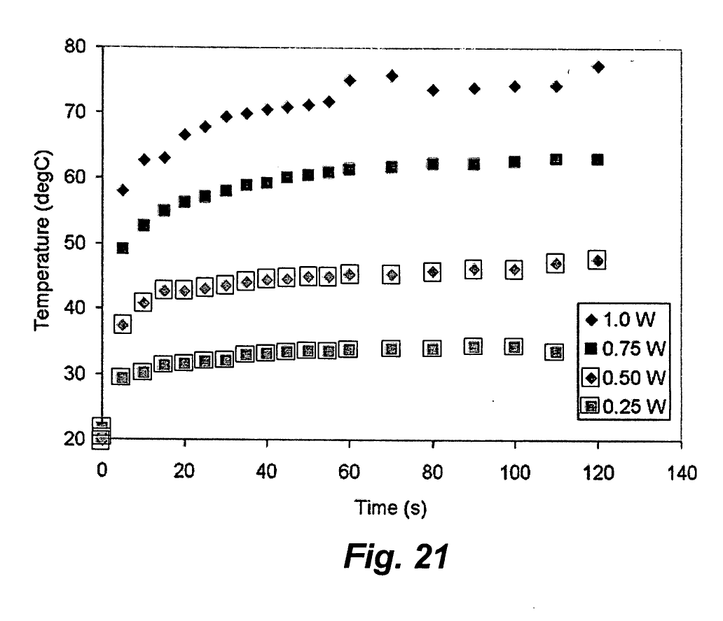

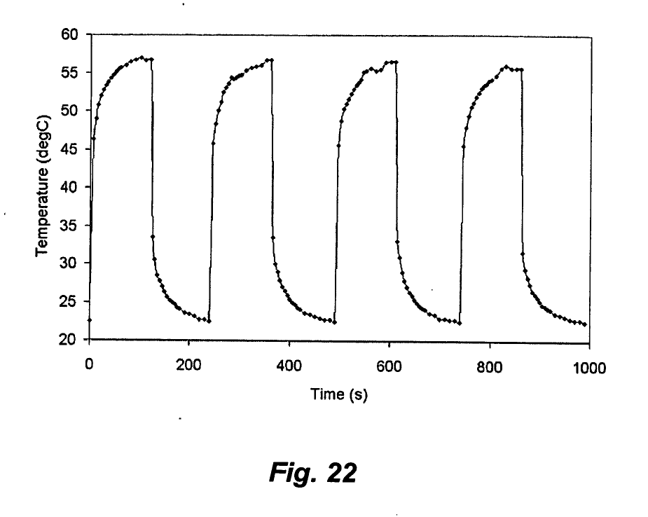

Further, FIG. 21 demonstrates an influence of laser power on the kinetics of the thermographic detection of nucleic acids. More particularly, the data provided in FIG. 21 show the rate at which the temperature can rise for a number of laser powers. These data demonstrate that the temperature change can be a function of the laser power.

Also, as shown in FIG. 22, the thermographic effect measured by the presently disclosed subject matter is reversible. More particularly, FIG. 22 shows the reversibility of four excitation cycles of the presently disclosed subject matter.

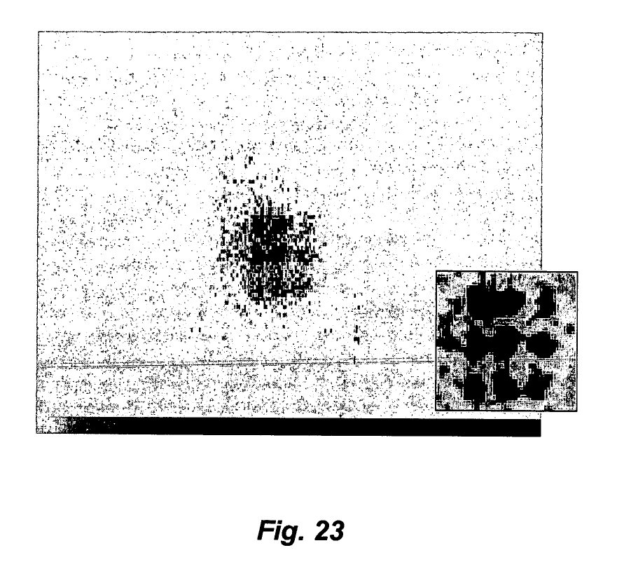

Finally, an array also can be read with thermography. FIG. 23 demonstrates the bloc reading of a 3×3 array. Rapid reading is provided, without needing to perfectly align the spot and beam.

It will be understood that various details of the presently disclosed subject matter can be changed without departing from the scope of the presently disclosed subject matter. Furthermore, the foregoing description is for the purpose of illustration only, and not for the purpose of limitation.

1. A method of detecting a target nucleic acid sequence, comprising:
providing a hybridization complex comprising (a) a capture probe that is attached to an electrode and (b) a target nucleic acid sequence that is hybridized to the capture probe, wherein the target nucleic acid sequence additionally comprises at least one nanoparticle attached to the target nucleic acid sequence;
exposing the electrode to light while the electrode is in contact with a redox solution, wherein the redox solution comprises a redox mediator and an electrolyte, and wherein the light has a wavelength absorbed by the nanoparticle; and
detecting an electrical signal in the electrode, whereby detection of an increased electrical signal relative to a signal that would be detected in the absence of said complex indicates the presence or amount of target nucleic acid sequence hybridized to the electrode.

2. The method of claim 1, comprising:
hybridizing a target sequence to at least one capture probe to form a first hybridization complex, wherein the capture probe is attached to an electrode;
hybridizing a detection probe to the first hybridization complex to form a second hybridization complex, wherein the detection probe comprises a nanoparticle;
exposing the electrode to light while the electrode is in contact with a redox solution, wherein the redox solution comprises a redox mediator and an electrolyte, and wherein the light has a wavelength absorbed by the nanoparticle; and
detecting the amount of electron transfer to the electrode, wherein an increase in electron transfer as compared to electron transfer to the electrode in the absence of detection probe indicates hybridization of the target sequence to the electrode.

3. The method of claim 1, wherein the target sequence comprises RNA.

4. The method of claim 1, wherein the target sequence comprises cDNA.

5. The method of claim 1, wherein the target sequence is present in a biological sample.

6. The method of claim 1, wherein the electrode comprises a conducting material comprising one or more of metals and metal oxides.

7. The method according to claim 1, wherein the electrode comprises indium tin oxide.

8. The method according to claim 1, wherein the electrode is formed on a non-conducting solid substrate.

9. The method according to claim 2, wherein the detection probe comprises a nanoparticle comprising a material comprising one or more of metals and metal oxides.

10. The method according to claim 9, wherein the nanoparticle comprises a metal comprising one or more of gold, silver, platinum and palladium.

11. The method according to claim 1, wherein the nanoparticle comprises gold.

12. The method according to claim 1, wherein the nanoparticle comprises silver.

13. The method according to claim 1, wherein the nanoparticle is a nanoshell.

14. The method according to claim 1, wherein the nanoparticle has a diameter from about 10 to about 20 nanometers.

15. The method according to claim 1, wherein the detection probe further comprises an oligonucleotide attached to the nanoparticle.

16. The method according to claim 15, wherein the capture probe is complementary to a first target domain of the target sequence, and the oligonucleotide component of the detection probe is complementary to a second target domain of the target sequence.

17. The method according to claim 1, wherein the detection probe comprises a nanoparticle attached to one partner of a ligand binding pair, and the target sequence comprises the other partner of a ligand binding pair.

18. The method according to claim 17, wherein one partner of a ligand binding pair is streptavidin, and the other partner of the ligand binding pair is biotin.

19. The method according to claim 17, wherein the target sequence comprises biotin.

20. The method according to claim 19, wherein the biotin has been incorporated into the target sequence during nucleic acid amplification.

21. The method according to claim 17, wherein the detection probe comprises a nanoparticle attached to streptavidin.

22. The method according to claim 1, wherein the redox mediator comprises a metallocene.

23. The method according to claim 1, wherein the redox mediator comprises ferrocene.

24. The method according to claim 1, wherein the redox mediator comprises EDTA.

25. The method according to claim 1, wherein the light is generated by a laser.

26. The method according to claim 1, wherein the detecting step is carried out by cyclic voltammetry.

27. The method according to claim 1, wherein the detecting step is carried out by chronoamperometry.

28. The method according to claim 1, wherein a plurality of different capture probes is attached to the electrode in an array, and the location of each capture probe comprises an attachment point.

29. The method according to claim 28, wherein each attachment point of the array is exposed to light separately.

30. The method according to claim 1, wherein the light is provided by a light source is selected from the group consisting of a tungsten halogen light source, a xenon arc lamp and a laser.

31. The method according to claim 1, where in the exposing is carried out by rastering.

32. The method according to claim 1, wherein the redox solution further comprises a sacrificial electron donor.

33. The method according to claim 32, wherein the sacrificial electron donor comprises EDTA.

34. The method according to claim 1, further comprising passivating the electrode with a passivation moiety before contacting the target sequence with the capture probe.

35. The method according to claim 1, wherein the target sequence is selected from the group consisting of an mRNA sequence derived from a sample and a cDNA sequence derived from a sample.

36. The method according to claim 1, wherein the capture probe comprises a sequence from a gene of interest.

37. The method according to claim 36, wherein the presence of electric current is indicative of hybridization complex formation and hybridization complex formation is indicative of gene expression or a gene expression level.

38. The method according to claim 37, wherein the capture probe comprises or is suspected to comprise a mutation to be detected

39. The method according to claim 1, wherein the target sequence comprises or is suspected to comprise a mutation to be detected

40. The method according to claim 1, wherein the nanoparticle comprises gold and the nanoparticle is exposed to light at a wavelength of about 532 nm.

41. The method according to claim 1, wherein the nanoparticle comprises silver and the nanoparticle is exposed to light at a wavelength of about from about 420 nm to about 460 nm.

42. The method according to claim 1, wherein the target sequence is present in a concentration of less than about 10 picomoles.

43. The method according to claim 1, wherein electron transfer between the nanoparticle and the electrode is detected.

44. The method according to claim 1, wherein electron transfer between the nanoparticle and the electrode is detected.

45. The method according to claim 1, wherein the nanoparticle is attached to the target sequence.

46. The method according to claim 45, where the nanoparticle is attached to the target sequence by one of a binding pair and complementary nucleic acids.

47. The method according to claim 45, where the nanoparticle is attached to the target sequence by one of primer extension and ligation of a nanoparticle-labeled nucleic acid.

48. The method of claim 1, wherein the complex comprises a detection probe.

49. The method of claim 48, wherein the detection probe is attached to the target sequence before, during, or after the target sequence hybridizes to the capture probe.

50. The method of claim 1, comprising the sequential steps of hybridizing the target to the capture probe; and then reacting the hybrid with a detection probe.
